SEQUENCE LISTING (93 sequences)

SEQ ID NO: 1 - DNA, 20 bp, synthetic construct
  source 1..20: mol_type = other DNA
gcctctcacttttgctcgga

SEQ ID NO: 2 - DNA, 20 bp, synthetic construct
  source 1..20: mol_type = other DNA
aggattgcactcaaaggccc

SEQ ID NO: 3 - DNA, 25 bp, synthetic construct
  source 1..25: mol_type = other DNA
ggctcgatatatcatgagtgattcc

SEQ ID NO: 4 - DNA, 25 bp, synthetic construct
  source 1..25: mol_type = other DNA
catgatatatcgagccaatccaaag

SEQ ID NO: 5 - DNA, 25 bp, synthetic construct
  source 1..25: mol_type = other DNA
ggctcgacatatcatgagtgattcc

SEQ ID NO: 6 - DNA, 25 bp, synthetic construct
  source 1..25: mol_type = other DNA
catgatatgtcgagccaatccaaag

SEQ ID NO: 7 - DNA, 25 bp, synthetic construct
  source 1..25: mol_type = other DNA
ggctcgagttatcatgagtgattcc

SEQ ID NO: 8 - DNA, 25 bp, synthetic construct
  source 1..25: mol_type = other DNA
catgataactcgagccaatccaaag

SEQ ID NO: 9 - DNA, 25 bp, synthetic construct
  source 1..25: mol_type = other DNA
ggctcgatttatcatgagtgattcc

SEQ ID NO: 10 - DNA, 25 bp, synthetic construct
  source 1..25: mol_type = other DNA
catgataaatcgagccaatccaaag

SEQ ID NO: 11 - DNA, 20 bp, synthetic construct
  source 1..20: mol_type = other DNA
ggtacctcctactgaagttg

SEQ ID NO: 12 - DNA, 20 bp, synthetic construct
  source 1..20: mol_type = other DNA
gtaagcagactgctgtgagg

SEQ ID NO: 13 - DNA, 32 bp, synthetic construct
  source 1..32: mol_type = other DNA
aaagtggtgaagatatgtgactttggattggc

SEQ ID NO: 14 - DNA, 30 bp, synthetic construct
  source 1..30: mol_type = other DNA
ggtgaagatatgtgactttggattggctcg

SEQ ID NO: 15 - DNA, 33 bp, synthetic construct
  source 1..33: mol_type = other DNA
ctccaggataatacacatcacagtaaataacac

SEQ ID NO: 16 - DNA, 34 bp, synthetic construct
  source 1..34: mol_type = other DNA
cacaacacaaaatagccgtataaaaataagtagg

SEQ ID NO: 17 - DNA, 33 bp, synthetic construct
  source 1..33: mol_type = other DNA
tttaccatgataacgacacaacacaaaatagcc

SEQ ID NO: 18 - DNA, 35 bp, synthetic construct
  source 1..35: mol_type = other DNA
ccttttaagcataagtaagcagactgctgtgaggg

SEQ ID NO: 19 - DNA, 20 bp, synthetic construct
  source 1..20: mol_type = other DNA
catgaagaattttaggaccc

SEQ ID NO: 20 - DNA, 20 bp, synthetic construct
  source 1..20: mol_type = other DNA
ccagcctcacctcgtcggtg

SEQ ID NO: 21 - DNA, 20 bp, synthetic construct
  source 1..20: mol_type = other DNA
attggcaagcacgcccatgg

SEQ ID NO: 22 - DNA, 20 bp, synthetic construct
  source 1..20: mol_type = other DNA
ggcgtgcttgccaatggtga

SEQ ID NO: 23 - DNA, 20 bp, synthetic construct
  source 1..20: mol_type = other DNA
agccataagtcctcgacgtg

SEQ ID NO: 24 - DNA, 20 bp, synthetic construct
  source 1..20: mol_type = other DNA
ctgcgagctcacccagaatg

SEQ ID NO: 25 - DNA, 22 bp, synthetic construct
  source 1..22: mol_type = other DNA
tttgggcgggccaaactgctgg

SEQ ID NO: 26 - DNA, 25 bp, synthetic construct
  source 1..25: mol_type = other DNA
tttggcccgcccaaaatctgtgatc

SEQ ID NO: 27 - DNA, 20 bp, synthetic construct
  source 1..20: mol_type = other DNA
aatggaaggtcacactaggg

SEQ ID NO: 28 - DNA, 20 bp, synthetic construct
  source 1..20: mol_type = other DNA
acagaatatgggtaaagatg

SEQ ID NO: 29 - DNA, 20 bp, synthetic construct
  source 1..20: mol_type = other DNA
ggagcagatggtgttgggaa

SEQ ID NO: 30 - DNA, 20 bp, synthetic construct
  source 1..20: mol_type = other DNA
caacaccatctgctccaacc

SEQ ID NO: 31 - DNA, 32 bp, synthetic construct
  source 1..32: mol_type = other DNA
tccctggctggaccaagcccatcacctttggc

SEQ ID NO: 32 - DNA, 30 bp, synthetic construct
  source 1..30: mol_type = other DNA
tgcccaggtcagtggatcccctctccaccc

SEQ ID NO: 33 - DNA, 34 bp, synthetic construct
  source 1..34: mol_type = other DNA
tcccagaaggtgagaaagttcaaattcccgtcgc

SEQ ID NO: 34 - DNA, 31 bp, synthetic construct
  source 1..31: mol_type = other DNA
ttcagagccatggacccccacacagcaaagc

SEQ ID NO: 35 - DNA, 30 bp, synthetic construct
  source 1..30: mol_type = other DNA
aacgtactggtgaaaacaccgcagcatgtc

SEQ ID NO: 36 - DNA, 30 bp, synthetic construct
  source 1..30: mol_type = other DNA
cacctccttactttgcctccttctgcatgg

SEQ ID NO: 37 - DNA, 34 bp, synthetic construct
  source 1..34: mol_type = other DNA
ttcttgctggtgtgaaatgactgagtacaaactg

SEQ ID NO: 38 - DNA, 33 bp, synthetic construct
  source 1..33: mol_type = other DNA
tctatggtgggatcatattcatctacaaagtgg

SEQ ID NO: 39 - RNA, 44 nt, synthetic construct
  source 1..44: mol_type = other RNA
taatttctactaagtgtagatgattggctcgatatatcatgagt

SEQ ID NO: 40 - RNA, 44 nt, synthetic construct
  source 1..44: mol_type = other RNA
taatttctactaagtgtagatgattggctcgatacatcatgagt

SEQ ID NO: 41 - RNA, 44 nt, synthetic construct
  source 1..44: mol_type = other RNA
taatttctactaagtgtagatgattggctctatatatcatgagt

SEQ ID NO: 42 - RNA, 44 nt, synthetic construct
  source 1..44: mol_type = other RNA
taatttctactaagtgtagatgattggctcgatatataatgagt

SEQ ID NO: 43 - RNA, 44 nt, synthetic construct
  source 1..44: mol_type = other RNA
taatttctactaagtgtagatgattggctcgacatatcatgagt

SEQ ID NO: 44 - RNA, 44 nt, synthetic construct
  source 1..44: mol_type = other RNA
taatttctactaagtgtagatgattggctcgacacatcatgagt

SEQ ID NO: 45 - RNA, 44 nt, synthetic construct
  source 1..44: mol_type = other RNA
taatttctactaagtgtagatgattggctcgagttatcatgagt

SEQ ID NO: 46 - RNA, 44 nt, synthetic construct
  source 1..44: mol_type = other RNA
taatttctactaagtgtagatgattggctcgcgttatcatgagt

SEQ ID NO: 47 - RNA, 44 nt, synthetic construct
  source 1..44: mol_type = other RNA
taatttctactaagtgtagatgattggctcgagttctcatgagt

SEQ ID NO: 48 - RNA, 44 nt, synthetic construct
  source 1..44: mol_type = other RNA
taatttctactaagtgtagatgattggctcgattcatcatgagt

SEQ ID NO: 49 - RNA, 44 nt, synthetic construct
  source 1..44: mol_type = other RNA
taatttctactaagtgtagatgattggctcgagatatcatgagt

SEQ ID NO: 50 - RNA, 44 nt, synthetic construct
  source 1..44: mol_type = other RNA
taatttctactaagtgtagatgattggctcgagacatcatgagt

SEQ ID NO: 51 - RNA, 44 nt, synthetic construct
  source 1..44: mol_type = other RNA
taatttctactaagtgtagatgattggctctagatatcatgagt

SEQ ID NO: 52 - RNA, 44 nt, synthetic construct
  source 1..44: mol_type = other RNA
taatttctactaagtgtagatgattggctcgagatataatgagt

SEQ ID NO: 53 - RNA, 44 nt, synthetic construct
  source 1..44: mol_type = other RNA
taatttctactaagtgtagatgcaggcgcgcccatggcgaccag

SEQ ID NO: 54 - RNA, 44 nt, synthetic construct
  source 1..44: mol_type = other RNA
taatttctactaagtgtagatgcaagcgcgcccatggcgaccag

SEQ ID NO: 55 - RNA, 44 nt, synthetic construct
  source 1..44: mol_type = other RNA
taatttctactaagtgtagatggagatgttgcttctcttaattc

SEQ ID NO: 56 - RNA, 44 nt, synthetic construct
  source 1..44: mol_type = other RNA
taatttctactaagtgtagatggagatgttttgatagcgacggg

SEQ ID NO: 57 - RNA, 44 nt, synthetic construct
  source 1..44: mol_type = other RNA
taatttctactaagtgtagatggctggctaaactgctgggtgcg

SEQ ID NO: 58 - RNA, 44 nt, synthetic construct
  source 1..44: mol_type = other RNA
taatttctactaagtgtagatggcgggctaaactgctgggtgcg

SEQ ID NO: 59 - RNA, 44 nt, synthetic construct
  source 1..44: mol_type = other RNA
taatttctactaagtgtagatccaacaccacccgctccaaccac

SEQ ID NO: 60 - RNA, 44 nt, synthetic construct
  source 1..44: mol_type = other RNA
taatttctactaagtgtagatccaacaccatccactccaaccac

SEQ ID NO: 61 - DNA, 26 bp, synthetic construct
  source 1..26: mol_type = other DNA
atcacgtcaccggtacctcctactga

SEQ ID NO: 62 - DNA, 26 bp, synthetic construct
  source 1..26: mol_type = other DNA
cgatgttcaccggtacctcctactga

SEQ ID NO: 63 - DNA, 28 bp, synthetic construct
  source 1..28: mol_type = other DNA
ttaggcagtcaccggtacctcctactga

SEQ ID NO: 64 - DNA, 27 bp, synthetic construct
  source 1..27: mol_type = other DNA
tgaccagtcaccggtacctcctactga

SEQ ID NO: 65 - DNA, 28 bp, synthetic construct
  source 1..28: mol_type = other DNA
acagtgcttcaccggtacctcctactga

SEQ ID NO: 66 - DNA, 26 bp, synthetic construct
  source 1..26: mol_type = other DNA
gccaattcaccggtacctcctactga

SEQ ID NO: 67 - DNA, 27 bp, synthetic construct
  source 1..27: mol_type = other DNA
cagatcttcaccggtacctcctactga

SEQ ID NO: 68 - DNA, 28 bp, synthetic construct
  source 1..28: mol_type = other DNA
acttgaaatcaccggtacctcctactga

SEQ ID NO: 69 - DNA, 27 bp, synthetic construct
  source 1..27: mol_type = other DNA
gatcaggtcaccggtacctcctactga

SEQ ID NO: 70 - DNA, 28 bp, synthetic construct
  source 1..28: mol_type = other DNA
tagcttcctcaccggtacctcctactga

SEQ ID NO: 71 - DNA, 26 bp, synthetic construct
  source 1..26: mol_type = other DNA
ggctactcaccggtacctcctactga

SEQ ID NO: 72 - DNA, 26 bp, synthetic construct
  source 1..26: mol_type = other DNA
cttgtatcaccggtacctcctactga

SEQ ID NO: 73 - DNA, 26 bp, synthetic construct
  source 1..26: mol_type = other DNA
agtcaatcaccggtacctcctactga

SEQ ID NO: 74 - DNA, 27 bp, synthetic construct
  source 1..27: mol_type = other DNA
agttccttcaccggtacctcctactga

SEQ ID NO: 75 - DNA, 28 bp, synthetic construct
  source 1..28: mol_type = other DNA
atgtcagctcaccggtacctcctactga

SEQ ID NO: 76 - DNA, 28 bp, synthetic construct
  source 1..28: mol_type = other DNA
ccgtccattcaccggtacctcctactga

SEQ ID NO: 77 - DNA, 27 bp, synthetic construct
  source 1..27: mol_type = other DNA
gtagagctcaccggtacctcctactga

SEQ ID NO: 78 - DNA, 27 bp, synthetic construct
  source 1..27: mol_type = other DNA
gtccgcatcaccggtacctcctactga

SEQ ID NO: 79 - DNA, 28 bp, synthetic construct
  source 1..28: mol_type = other DNA
gtgaaatctcaccggtacctcctactga

SEQ ID NO: 80 - DNA, 26 bp, synthetic construct
  source 1..26: mol_type = other DNA
gtggcctcaccggtacctcctactga

SEQ ID NO: 81 - DNA, 26 bp, synthetic construct
  source 1..26: mol_type = other DNA
gtttcgtcaccggtacctcctactga

SEQ ID NO: 82 - DNA, 27 bp, synthetic construct
  source 1..27: mol_type = other DNA
cgtacggtcaccggtacctcctactga

SEQ ID NO: 83 - DNA, 28 bp, synthetic construct
  source 1..28: mol_type = other DNA
gagtggagtcaccggtacctcctactga

SEQ ID NO: 84 - DNA, 27 bp, synthetic construct
  source 1..27: mol_type = other DNA
ggtagcatcaccggtacctcctactga

SEQ ID NO: 85 - DNA, 27 bp, synthetic construct
  source 1..27: mol_type = other DNA
actgatgtcaccggtacctcctactga

SEQ ID NO: 86 - DNA, 28 bp, synthetic construct
  source 1..28: mol_type = other DNA
atgagccatcaccggtacctcctactga

SEQ ID NO: 87 - DNA, 26 bp, synthetic construct
  source 1..26: mol_type = other DNA
attccttcaccggtacctcctactga

SEQ ID NO: 88 - DNA, 28 bp, synthetic construct
  source 1..28: mol_type = other DNA
caccggcatcaccggtacctcctactga

SEQ ID NO: 89 - DNA, 22 bp, synthetic construct
  source 1..22: mol_type = other DNA
gaaatagcagcctcacattgcc

SEQ ID NO: 90 - DNA, 23 bp, synthetic construct
  source 1..23: mol_type = other DNA
cgggaaagtggtgaagatatgtg

SEQ ID NO: 91 - DNA, 27 bp, synthetic construct
  source 1..27: mol_type = other DNA
ctgacaacatagttggaatcactcatg

SEQ ID NO: 92 - DNA, 18 bp, synthetic construct
  source 1..18: mol_type = other DNA
ctcgagatatcatgagtg

SEQ ID NO: 93 - DNA, 19 bp, synthetic construct
  source 1..19: mol_type = other DNA
ttggattggctcgagatat

(19) United States
(12) Patent Application Publication
(10) Pub. No.: US 2025/0019753 A1
(43) Pub. Date: Jan. 16, 2025

(54) METHODS FOR NUCLEIC ACID DETECTION

(71) Applicant: AGRICULTURAL GENOMICS INSTITUTE AT SHENZHEN, CHINESE ACADEMY OF AGRICULTURAL SCIENCES, SHENZHEN (CN)

(72) Inventor: Xinjie WANG, SHENZHEN (CN)

(73) Assignee: AGRICULTURAL GENOMICS INSTITUTE AT SHENZHEN, CHINESE ACADEMY OF AGRICULTURAL SCIENCES, SHENZHEN (CN)

(21) Appl. No.: 18/711,870

(22) PCT Filed: Nov. 21, 2022
(86) PCT No.: PCT/CN2022/133119

§ 371 Date: May 20, 2024

(30) Foreign Application Priority Data
Nov. 20, 2021 (WO) PCT/CN2021/131956

(51) Int. Cl. C12Q 1/6848

(52) U.S. Cl. CPC C12Q 1/6848

(57) ABSTRACT

The present disclosure provides methods, kits, and materials for amplifying, enriching or detecting a target nucleic acid, especially in a small amount, in a sample by digesting or degrading non-target nucleic acids in the sample before or during amplification and may detect the target nucleic acids. Interference of the non-target nucleic acids before or during amplification or detection will be minimized and the sensitivity, accuracy and efficiency of the amplification or detection will be greatly increased.

### FIELD

The disclosure relates to methods for nucleic acid detection.

### BACKGROUND

Sensitive, accurate and efficient detection of nucleic acid sequence variants is essential for precision medicine, where the individualized treatment is provided based on the unique genetic profile of each patient. However, profiling rare DNA or RNA variants with low allele frequencies in cancer samples has challenged current molecular diagnostic technologies for a long time (Khodakov D, et al. Adv Drug Deliv Rev 105, 3-19 (2016). [PubMed: 27089811]). The first-generation sequencing (FGS) approaches are not sensitive enough to detect a mutation rate under 10%. The next-generation sequencing (NGS) approaches are time-consuming and not economical. Allele-specific PCR methods are prone to artificially introduced mutations, while the specificity of qPCR-based methods depends excessively on the primers and the probes. Detection sensitivity of PCR-based methods can be enhanced by restriction digestion of wild-type (WT) sequences in the sample to enrich target sequences, but only at a cost of much complicated workflow (Zhao A H, et al. J Hematol Oncol 4, 40 (2011). [PubMed: 21985400]).

CRISPR-based gene editing systems have shown great potential for rapid and sensitive nucleic acid detection, including those based on Cas9, Cas12, and Cas13. Recently, Cas12-or Cas13-based detection has been applied to SARS-COV-2 diagnosis in coordination with isothermal amplification, which proved highly effective because the samples have little contaminants from host nucleic acid. In sharp contrast, for detection of rare genetic variants and mutations, the majority of the DNA or RNA are WT sequences, which significantly hampers the analysis.

Xianfeng, WANG et al. (Dual Methylation-Sensitive Restriction Endonucleases Coupling with an RPA-Assisted CRISPR/Cas13a System (DESCS) for Highly Sensitive Analysis of DNA Methylation and Its Application for Point-of-Care Detection, ACS Sens. 2021, 6, 2419-2428) disclosed dual methylation-sensitive restriction endonucleases (BstUI/HhaI) coupling with an RPA-assisted CRISPR/Cas 13a system for site-specific methylation detection of the SEPT9 gene. However, only the SEPT9 gene was tested in this article. It is unable to conclude that this method is applicable to detect any methylation(s) in any sequence of interest. Further, BstUI/HhaI used in this article are only sensitive to DNA CpG methylation in genome of a mammal, but not to Dam methylation or Dem methylation. And only Cas13a was used in the detection. Methods for sensitively, and simply detecting mutations, such as deletion, insertion and/or substituent in a nucleic acid of interest, were not disclosed in this article and remains elusive.

### SUMMARY

In general, provided are methods, kits, and materials for amplifying, enriching or detecting a target nucleic acid, especially in a small amount, in a sample by digesting or degrading non-target nucleic acids in the sample before or during amplification and may detect the amplified target nucleic acids.

In some embodiments, the disclosure provides a method for amplifying or enriching target nucleic acids with alternation(s) of interest at a specified site in a sample, including digesting or degrading non-target nucleic acids without the alternation(s) of interest at the specified site in the sample by exposing the non-target nucleic acids to one or more proteins having an activity of cleaving nucleic acid that recognizes the base(s) at the specified site before or during amplification.

In some embodiments, the disclosure provides a method for detecting a target nucleic acid with alternation(s) of interest at a specified site in a sample, including digesting or degrading non-target nucleic acids without the alternation(s) of interest at the specified site in the sample by exposing the non-target nucleic acids to one or more proteins having an activity of cleaving nucleic acid that recognize the specified site before or during amplification, and detecting the amplified target nucleic acid.

In one or more embodiments, the protein having an activity of cleaving nucleic acid is selected from the group consisting of: a restriction endonuclease, a Cas enzyme, an Ago enzyme, a ZFN enzyme, a TALEN enzyme, and a functional complex thereof, such as Cas enzyme/sgRNA complex, Ago/gDNA complex, or Cas12a/crRNA complex. Preferably, the Cas enzyme/sgRNA complex is a Cas9/sgRNA complex, more preferably, a spCas9/sgRNA complex. Preferably, the Ago/gDNA complex is a pfAgo/gDNA complex. Preferably, the Cas12a/crRNA complex is a LbCas 12a/crRNA complex.

In one or more embodiments, the proteins having an activity of cleaving nucleic acid is a restriction endonuclease, and the non-target nucleic acid contains the recognizing site of the restriction endonuclease at the specified site.

In one or more embodiments, the restriction endonuclease is selected from the group consisting of BclI, BsaBI, AclWI, Bst4CI, Xmil, BlsI, PspFI, CviKI-1, CviJI, MscI, EcoP15I, BspACI, BseLI, BstKTI, PspN4I, BspLI, NlaIV, Bmil, MaiI, RsnAI, BspEI, HpaII, Mrol, Kpn2I, BcoDI, BstDEI, BpulOI, BtsIMutI, BasBI, BtsCI, NspI, Fail, EcoRV and Btsal.

In one or more embodiments, the Ago enzyme is selected from a group consisting of pfAgo, cbAgo, LrAgo, pfAgo-mut, Apol, pfAgo, TtAgo and MjAgo.

In one or more embodiments, the protein having an activity of cleaving nucleic acid is a Cas enzyme, and a gRNA having a targeting region which binds to the non-target nucleic acid sequence at the specified site is used to direct the Cas enzyme cleavage of the nucleic acid sequence.

In one or more embodiments, the Cas enzyme is selected from the group consisting of Cas 9, Cas 12, Cas 13 and Cas 14, especially SpCas9, SaCas9, HypaCas9, StlCas9, spCas9-NG, LbCas12a, spCas9-mut, and ScCas9.

In one or more embodiments, exposing the non-target nucleic acids to the one or more proteins having an activity of cleaving nucleic acid is performed by adding the protein(s) in an amplification mixture for amplifying the target nucleic acids.

In one or more embodiments, the amplification is selected from the group consisting of: helicase-dependent amplification (HAD), polymerase chain reaction (PCR), DNA ligase chain reaction (LCR), isothermal DNA amplification, QBeta RNA replicase, RNA transcription-based amplification reactions, loop-mediated isothermal amplification (LAMP), RT-LAMP, recombinase polymerase amplification (RPA), reverse transcription-recombinase polymerase amplification (RT-RPA), helicase-dependent amplification (HDA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification

(SPIA), signal mediated amplification of RNA technology (SMART), self-sustained sequence replication (3SR), genome exponential amplification reaction (GEAR), and isothermal multiple displacement amplification (IMDA).

In one or more embodiments, the amplification mixture is an isothermal nucleic acid amplification mixture.

In one or more embodiments, the amplification is RPA.

In one or more embodiments, the alternation includes deletion, substitution and insertion of one or more base(s) at the specified site as compared to the non-target nucleic acid.

In one or more embodiments, the alternation is alternation of two or more continuous bases as compared to the non-target nucleic acid.

In one or more embodiments, detection of the amplified target nucleic acid indicates the presence of the alternation in the subject.

In one or more embodiments, the target nucleic acid is detected by DNA staining, nucleic acid amplification, spectrophotometry, sequencing, fluorescent probe hybridization, fluorescence resonance energy transfer, optical microscopy, electron microscopy, CRISPR-based detection, visual based detection, sensor-based detection, color detection, gold nanoparticle based detection, electrochemical detection, semiconductor-based sensing or combinations thereof.

In one or more embodiments, the amplified target nucleic acid is detected with one or more protein(s) capable of recognizing a specific nucleic acid sequence, or a functional complex thereof.

In one or more embodiments, the protein(s) capable of recognizing a specific nucleic acid sequence include Cas enzyme, Ago enzyme, ZFN enzyme, TALEN enzyme, and functional complexes thereof.

In one or more embodiments, the Cas enzyme is selected from the group consisting of Cas 9, Cas 12, Cas 13 and Cas 14, especially includes but is not limited to SpCas9, SaCas9,HypaCas9, StlCas9, spCas9-NG, LbCas 12a, spCas9-mut, and ScCas9.

In one or more embodiments, the Ago enzyme is selected from the group consisting of pfAgo, cbAgo, LrAgo, pfAgo-mut, Apol, pfAgo, TtAgo and MjAgo.

In one or more embodiments, the functional complex is selected from the group consisting of Cas enzyme/sgRNA complex, Ago/gDNA complex and Cas12a/crRNA complex: more preferably, the Cas enzyme/sgRNA complex is a Cas9/sgRNA complex, more preferably, a spCas9/sgRNA complex: the Ago/gDNA complex is a pfAgo/gDNA complex: the Cas12a/crRNA complex is a LbCas12a/crRNA complex.

In one or more embodiments, the protein capable of recognizing a specific nucleic acid sequence is selected from the group consisting of LbCas12a, FnCas 12a, Lb5Cas12a, HkCas12a, TsCas12a, BbCas12a, BoCas12a, Lb4Cas12a, LbuCas13a, LwaCas13a, LbaCas13a, PprCas13a, HheCas13a, EreCas13a, AsCas12a, TsCas12a, BbCas12a, BoCas12a, Lb4Cas12a, spCas9, pfAgo, cbAgo, LrAgo, Cas12b, Cas12a-mut, Cas 12b-mut, AapCas 12b, BrCas12b, CcaCas13b, PsmCas13b and AacCas12b, or functional complexes thereof.

In one or more embodiments, digesting or degrading and detecting are carried out sequentially or simultaneously.

In one or more embodiments, digesting or degrading and detecting are carried out in the same reaction system.

In yet another embodiment, the disclosure provides a kit for amplifying or enriching and may detect target nucleic acids with alternation(s) of interest at a specified site in a sample, including reagents for amplification of target nucleic acids with alternation(s) of interest at the specified site in the sample, and reagents for digesting non-target nucleic acids without the alternation(s) of interest at the specified site in the sample.

In one or more embodiments, the alternation includes deletion, substitution and/or insertion of one or more base(s) at the specified site as compared to the non-target nucleic acid.

In one or more embodiments, the alternation is alternation of two or more continuous bases as compared to the non-target nucleic acid.

In one or more embodiments, the reagents for digesting non-target nucleic acids include a protein having an activity of cleaving nucleic acid.

In one or more embodiments, the protein having an activity of cleaving nucleic acid is selected from the group consisting of: a restriction endonuclease, a Cas enzyme, an Ago enzyme, a ZFN enzyme, a TALEN enzyme, and a functional complex thereof, such as Cas enzyme/sgRNA complex, Ago/gDNA complex, or Cas 12a/crRNA complex. Preferably, the Cas enzyme/sgRNA complex is a Cas9/sgRNA complex, more preferably, a spCas9/sgRNA complex. Preferably, the Ago/gDNA complex is a pfAgo/gDNA complex. Preferably, the Cas12a/crRNA complex is a LbCas 12a/crRNA complex.

In one or more embodiments, the proteins having an activity of cleaving nucleic acid is a restriction endonuclease, preferably is selected from the group consisting of BclI, BsaBI, AclWI, Bst4CI, Xmil, BlsI, PspFI, CviKI-1, CviJI, Mscl, EcoP151, BspACI, BseLI, BstKTI, PspN4I, BspLI, NlaIV, Bmil, MaiI, RsnAI, BspEI, HpaII, Mrol, Kpn2I, BcoDI, BstDEI, Bpu1OI, BtsIMutI, BasBI, BtsCI, NspI, Fail, EcoRV and Btsal.

In one or more embodiments, the proteins having an activity of cleaving nucleic acid is a Ago enzyme, preferably selected from a group consisting of pfAgo, cbAgo, LrAgo, pfAgo-mut, Apol, pfAgo, TtAgo and Mj Ago.

In one or more embodiments, the protein having an activity of cleaving nucleic acid is a Cas enzyme, preferably is selected from the group consisting of Cas 9, Cas 12, Cas 13 and Cas 14, especially SpCas9, SaCas9, HypaCas9, StlCas9, spCas9-NG, LbCas12a, spCas9-mut, and ScCas9.

In one or more embodiments, the reagents for digesting non-target nucleic acids include restriction endonucleases, or Cas enzymes and a guide RNA having targeting region which binds to the non-target nucleic acid.

In one or more embodiments, the reagents for amplification of target nucleic acids with alternation(s) of interest at the specified site in the sample include reagent(s) used to perform any of helicase-dependent amplification (HAD), polymerase chain reaction (PCR), DNA ligase chain reaction (LCR), isothermal DNA amplification, QBeta RNA replicase, RNA transcription-based amplification reactions, loop-mediated isothermal amplification (LAMP), RT-LAMP, recombinase polymerase amplification (RPA), reverse transcription-recombinase polymerase amplification (RT-RPA), helicase-dependent amplification (HDA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), and isothermal multiple displacement amplification (IMDA).

In one or more embodiments, the reagents for amplification include reagents for PCR or isothermal amplification reaction. More preferably, the reagents for amplification include reagents used for RPA.

In one or more embodiments, the kit further includes reagents used for detecting the target nucleic acids, such as reagents used for DNA staining, nucleic acid amplification, spectrophotometry, sequencing, fluorescent probe hybridization, fluorescence resonance energy transfer, optical microscopy, electron microscopy, CRISPR-based detection, visual based detection, sensor based detection, color detection, gold nanoparticle based detection, electrochemical detection, semiconductor-based sensing or combinations thereof.

In one or more embodiments, the reagent(s) used for detecting the target nucleic acids include one or more protein(s) capable of recognizing a specific nucleic acid sequence, or a functional complex thereof.

In one or more embodiments, the protein(s) capable of recognizing a specific nucleic acid sequence include Cas enzyme, Ago enzyme, ZFN enzyme, TALEN enzyme, and functional complexes thereof.

In one or more embodiments, the Cas enzyme is selected from the group consisting of Cas 9. Cas 12, Cas 13 and Cas 14, especially includes but is not limited to SpCas9, SaCas9, HypaCas9, StlCas9, spCas9-NG, LbCas 12a, spCas9-mut, and ScCas9.

In one or more embodiments, the Ago enzyme is selected from the group consisting of pfAgo, cbAgo, LrAgo, pfAgo-mut, Apol, pfAgo. TtAgo and MjAgo.

In one or more embodiments, the functional complex is selected from the group consisting of Cas enzyme/sgRNA complex, Ago/gDNA complex and Cas12a/crRNA complex; more preferably, the Cas enzyme/sgRNA complex is a Cas9/sgRNA complex, more preferably, a spCas9/sgRNA complex; the Ago/gDNA complex is a pfAgo/gDNA complex; the Cas12a/crRNA complex is a LbCas12a/crRNA complex.

In one or more embodiments, the protein capable of recognizing a specific nucleic acid sequence is selected from the group consisting of LbCas12a, FnCas12a, Lb5Cas12a, HkCas12a, TsCas12a, BbCas12a, BoCas12a, Lb4Cas12a, LbuCas13a, LwaCas13a, LbaCas13a, PprCas13a, HheCas13a, EreCas13a, AsCas12a, TsCas12a, BbCas12a, BoCas12a, Lb4Cas12a, spCas9, pfAgo, cbAgo, LrAgo, Cas12b, Cas12a-mut, Cas12b-mut, AapCas12b, BrCas12b, CcaCas13b, PsmCas13b and AacCas12b, or functional complexes thereof.

In one or more preferred embodiments, the kit contains a mixture including reagents for RPA and the reagents for digesting the nucleic acid that do not contain the alteration. In some embodiments, the mixture also includes reagents used for detecting the target nucleic acids.

In one or more embodiments, the kit includes the kit includes the protein for cleavage listed in any one of the ID No. in Table A and reagent(s) for the amplification method listed in the same ID No., and includes the protein for detection listed in the same ID No. for detection.

### BRIEF DESCRIPTION OF THE DRAWINGS

FIG. 1 illustrates development of a method herein for sensitive mutation detection. a, Schemes of a detection method herein compared with CRISPR detection. b, The genomic location of FLT3-D835 mutation site and the proportions of different FLT3-D835 mutation types in AML patients in cBioportal database. c, Activity and specificity test of EcoRV digestion in its specific buffer. d, Activity and specificity test of EcoRV digestion in RPA mixture without primers. e, Fluorescence intensity of (d) after Cas12a reaction for 30 minutes.

FIG. 2 shows results of enhancing a method herein with optimized crRNAs and primers. a, Sequences of D835Y-crRNAs, and FLT3-WT and D835Y gene region. Base G mutates to T in D835Y. b, Fluorescence heatmap of different D835Y-crRNA-induced Cas12a reaction detecting 1 e10 copies of PCR fragments with different D835Y mutation rates, Cas12a reactions for 10, 20 and 30 minutes were recorded. c, Comparison of D835Y-crRNAs on the detection of 100% D835Y and WT samples. The chosen crRNA should have both high D835Y DNA induced fluorescence intensity while low WT DNA induced fluorescence intensity. d, Specificity assay of D835Y-crRNA2, D835H-crRNA, D835V-crRNA, and D835F-crRNA. Time-course of fluorescence intensity and naked-eye observation after 60 min of Cas12a reaction are shown. e, Schematic diagram of MMT-crRNAs-guided Cas12a reaction to identify D835Y/H/V/F mutations from WT background. f, Relative locations of designed RPA primers to the D835 site. g, A screen of RPA primer pairs for efficient amplification of the D835 region. The tested sample was 1e2 copies of 100% D835Y plasmid templates. And the reaction conditions were standard RPA for 20 minutes without EcoRV digestion, and MMT-crRNA-induced Cas12a reaction for 20 minutes, both under 37° C. h, Sensitivity assay of F2R1 mediated RPA combined with MMT-crRNAs induced Cas12a reaction, using gradient copies of D835Y plasmid templates as tested samples. Fluorescence intensity after 20 min of Cas12a reaction was shown. i, Time-course analysis of the detection of 1e1 D835Y plasmid templates by F2R1 mediated RPA combined with MMT-crRNAs induced Cas12a reaction.

FIG. 3 shows that a method herein achieves 0.001% sensitivity in D835Y mutation detection. a-b, Sensitivity comparison of CRISPR detection and a method herein in detecting le6 copies of plasmid templates with gradient D835Y mutation rates. c-d, Detection of the same amplified products in (a) using WT-crRNA-induced Cas12a reaction. e-f, FGS results of the amplified products in CRISPR detection and a method herein. The D835Y mutation rates were quantified using the online tool EditR (https://moriaritylab.shinyapps.io/editr v10/). g, the design and amplification plot of D835Y-probe 1-involved qPCR in detecting 1e6 copies of plasmid templates with gradient D835Y mutation rates. h, Ct value comparison of different samples.

FIG. 4 illustrates that a method herein accurately detect FLT3-D835Y/V/H/F mutations in clinical samples. a, Schematic diagram of mutation detection of AML patient cells by a method herein. About 1e6 cells was released by 100 ul nucleic acid releaser at 95° C. for 3 min, then 2 ul product was detected using a method herein, which includes EcoRV-integrated RPA at 37° C. for 20 minutes and MMT-crRNA-guided Cas12a reaction at 37° C. for 20 minutes. Positive result with a green fluorescence signal means that the sample has D835 mutation. b, NGS results of the FLT3-D835 mutation status of 32 AML samples, and their AML classification information. c, a method herein and FGS results of the 32 AML samples in the detection of FLT3-D835Y/V/H/F mutations. Patients with mutations are marked by red IDs and red boxes, and red triangles indicate mutant bases.

FIG. 5 shows that a method herein enables complete clinical diagnosis of FLT3-D835 mutations in an hour. a, Schematic diagram of the whole mutation diagnosis. The process from drawing blood to giving report can be completed in 1 hour. b, Equipment needed in a method herein. c, Detection results of drug-resistant FLT3-D835Y/V/H/F mutations of 80 AML patients using a method herein, FGS, and NGS, respectively. For FGS results, mutation bases are underlined and pointed out by red triangles. For NGS results, WT and mutant bases are colored green and red, respectively, and the numbers indicate their proportions. d, Statistical table of the sensitivity and specificity of a method herein compared with FGS using NGS as a standard reference.

FIG. 6 shows broad application of a method herein in cancer mutation diagnosis. a, Sensitivity comparison between a method herein and CRISPR detection in the detection of IDH2-R172K, EGFR-e19de1 and L858R, and NRAS-G12D mutations. Genomic locations of these mutations were shown above, and exons and mutation sites were colored in blue and red, respectively. The tested samples were 1e5 copies of plasmid templates with a mutation rate of 1% and 0.1%, respectively. Each amplified product was detected by both WT-crRNA and mutation-crRNA induced Cas12a reaction. Fluorescence intensity and naked eye results were both recorded. b, Statistic analysis of the MT/WT fluorescence ratio in a method herein and CRISPR detection. The results of 1% and 0.1% mutated samples were counted together. c-e, the qPCR assay for EGFR-el9del, L858R and NRAS-G12D detection, respectively. The qPCRs were performed on 10%, 1% and 0.1% mutated templates. A 100% WT template and ddH₂O (NC) served as control.

FIG. 7 shows the time-course analysis of FLT3-D835Y-crRNA1-4 guided Cas12a specific detection of the target PCR fragments with D835Y (GAT>TAT) mutation rate of 100%, 50%, 10%, 0% (WT), respectively. cr, crRNA; NC, negative control.

FIG. 8 shows the time-course analysis of FLT3-D835Y-crRNA2 induced Cas12a specific reaction with the target FLT3-D835Y, WT, and negative control fragments.

FIG. 9 illustrates optimization of FLT3-D835H-crRNA by an introduced mismatch. a, Sequences (brown) and target (purple) of FLT3-D835H-crRNA1 and crRNA2. The mutation base (GAT>CAT) and the introduced mismatch (U>C) are colored in red and orange, respectively. b, Time-course analysis of FLT3-D835H-crRNA1 and crRNA2 to detect a mutation between mutant (MTD835H) and WT allele. c, Comparison of fluorescence intensity after 60 min reaction.

FIG. 10 illustrates optimization of FLT3-D835V-crRNA by an introduced mismatch. a, Sequences (brown) and target (purple) of FLT3-D835V-crRNA1-3. The mutation base (GAT>GTT) and the introduced mismatches are colored in red and orange, respectively. b, Time-course analysis of FLT3-D835V-crRNA1 ˜3 to detect a mutation between mutant (MTD835V) and WT allele. c, Comparison of fluorescence intensity after 60 min reaction.

FIG. 11 shows specificity test of FLT3-D835F-crRNA. a, Sequences (brown) and target (purple) of FLT3-D835F-crRNA. The mutation bases (GAT>TTT) and an introduced mismatch are colored in red and orange, respectively. b, Time-course analysis of FLT3-D835F-crRNA to detect the mutation between mutant (MTD835F) and WT allele.

FIG. 12 shows optimization of FLT3-D835-WT-crRNA by an introduced mismatch. a, Sequences (brown) and target (purple) of FLT3-D835-WT-crRNA1-4. The mutation bases of D835Y, D835H, D835V, and D835F are colored in red, and the introduced mismatches are colored in orange. b, Comparison of fluorescence intensity after 60 min reaction, MMT=mixed-D835Y&H&V&F mutations. c, Time-course analysis of FLT3-D835-WTcrRNA1 ˜4 to detect mutations between WT and mutant allele.

FIG. 13 illustrates sequences and positions of mutation detection target and internal control (IC) target on FLT3 exon20, as well as the sequence of IC-crRNA.

FIG. 14 shows specificity assay of MMT-crRNAs. a, Specificity assay of MMT-crRNAs using 1e10 copies of D835Y/H/V/F and WT fragments. Photos were taken after 60 min of Cas12a reaction under a blue lamp. And the fluorescence intensity statistics are shown in b.

FIG. 15 illustrates sequences and locations of RPA primers for the amplification of FLT3-D835 region.

FIG. 16 shows time-course analysis of RPA primers screen for the amplification of FLT3-D835 region.

FIG. 17 shows sensitivity analysis of a detection system herein. a, Time-course analysis of MMT-crRNAs induced detection with 1E7-1E1 copies of D835Y plasmids. NC=negative control. b, Time-course record.

FIG. 18 shows WT inhibition assay by RPA with or without EcoRV and WT-crRNA induced Cas12a reaction, using 1E6-1E1 copies of WT plasmid templates. a, Fluorescence intensity statistics and naked-eye results. b, Time-course analysis.

FIG. 19 shows detection of 1E6-1E1 copies of D835Y and WT plasmids using RPA with or without EcoRV combined with MMT-crRNA induced Cas12a reaction. Histograms show the final fluorescence intensity.

FIG. 20 illustrates design of TaqMan qPCR for the detection of FLT3-D835Y. The forward primer, reverse primer and TaqMan probes are colored in orange, purple, and green, respectively.

FIG. 21 illustrates the amplification plot of D835Y-probe 1-involved qPCR in the detection of 1e5 copies of plasmid templates with gradient D835Y mutation rates.

FIG. 22 illustrates the amplification plot of D835Y-probe 2-involved qPCR in the detection of 1e5 copies of plasmid templates with gradient D835Y mutation rates.

FIG. 23 shows a method herein results (a) and next-generation sequencing (NGS) results (b) of 32 AML patient cell samples. Only three bases of D835 site are showed in NGS results, wild-type bases and mutated bases are colored in green and red, respectively.

FIG. 24 shows exploration of the fastest blood processing time before a detection method herein. Briefly, peripheral blood drawn from a patient with D835Y mutation was equally divided into 300 μl/sample, then mixed with 1200 μl red blood cell (RBC) lysis buffer. Different lysis time (0˜10 min) was used to screen the fastest condition. Then white blood cells (WBC) were collected by centrifugal precipitation for Imin, processed by nucleic acid releaser and detected using a method herein.

FIG. 25 shows a method herein results of 80 AML patient samples read by naked eyes under a 485 nm blue lamp.

FIG. 26 shows FGS results of the amplified products of a method herein and CRISPR detection. The tested samples were 1e5 copies of plasmid templates with a mutation rate of 1%. Mutated bases are pointed out by green and gray triangles, and the percentages indicate the mutation rates.

### DETAILED DESCRIPTION

### I. Definition

As used herein, the following terms have the meanings ascribed to them unless specified otherwise.

The term “endonuclease activity” refers to an enzyme activity of cleaving a polynucleotide chain by separating nucleotides other than the two end ones.

“The protein having an activity of cleaving nucleic acid” targets a nucleic acid and digests the nucleic acid by recognizing a certain site (i.e., certain short sequence) in the nucleic acid and then cleaving the nucleic acid. The recognizing site and the cleaving site can be the same or different in the nucleic acid. The protein having an activity of cleaving nucleic acid includes but is not limited to a restriction endonuclease, a Cas enzyme, an Ago enzyme, a ZFN enzyme, a TALEN enzyme, and a functional complex thereof.

A “functional complex” of a protein, such as an endonuclease, may include the endonuclease per se and molecule(s) capable of assisting the endonuclease to function. For example, a sgRNA or crRNA may be necessary for a Cas enzyme to function as a endonuclease. This is well known in the art. Therefore, for example, a functional complex as used herein include but is not limited to Cas enzyme/sgRNA complex, Ago/gDNA complex, or Cas12a/crRNA complex. Preferably, the Cas enzyme/sgRNA complex is a Cas9/sgRNA complex, more preferably, a spCas9/sgRNA complex. Preferably, the Ago/gDNA complex is a pfAgo/gDNA complex. Preferably, the Cas 12a/crRNA complex is a LbCas12a/crRNA complex. When referring to nucleic acids herein, “loci”, “site” or “position” can be used interchangeable.

The term “polymerase” refers to an enzyme that performs template-directed synthesis of polynucleotides by addition of nucleotide units to a nucleotide chain using DNA or RNA as a template. The term encompasses both a full length polypeptide and a domain that has polymerase activity. DNA polymerases are well-known in the art, and include but are not limited to DNA polymerases isolated or derived from Pyrococcus furiosus, Thermococcus litoralis, and Thermotoga maritime, Bacteriophage T4 or modified versions thereof.

“Thermally stable polymerase” as used herein, refers to any enzyme that catalyzes polynucleotide synthesis through thermal cycling. “Isothermal polymerase” as used herein, refers to any enzyme that catalyzes polynucleotide synthesis at a constant temperature (e.g., 37-42° C.) without thermal cycling, such as DNA recombinase polymerase derived from Bacteriophage T4.

The term “nucleic acid amplification” or “amplification reaction” refers to any in vitro means for multiplying the copies of a target sequence of nucleic acid. Such means include but are not limited to polymerase chain reaction (PCR), DNA ligase chain reaction (LCR), isothermal DNA amplification, QBeta RNA replicase, and RNA transcription-based amplification reactions as well as others known in the art. Particularly, such means include but are not limited to: loop-mediated isothermal amplification (LAMP), recombinase polymerase amplification (RPA), helicase-dependent amplification (HDA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), or isothermal multiple displacement amplification (IMDA).

“Amplifying” refers to a step of submitting a solution to conditions sufficient to allow for amplification of a polynucleotide. Components of an amplification reaction include, e.g., primers, a polynucleotide template, polymerase, nucleotides, and the like. The term amplifying typically refers to an “exponential” increase in target nucleic acid. However, amplifying as used herein can also refer to linear increases in the numbers of a select target sequence of nucleic acid, such as is obtained with cycle sequencing. In isothermal DNA amplification, the reaction also contains single stranded DNA binding (SSB). In one or more embodiments of the disclosure, the reaction also contains a protein having an activity of cleaving nucleic acid (such as restriction endonucleases, a Cas enzyme, an Ago enzyme, a ZFN enzyme, a TALEN enzyme, and a functional complex thereof).

“Isothermal DNA amplification” can be performed at a constant temperature without thermal cycling, including but not limited to: nucleic acid sequence-based amplification (NASBA), strand displacement amplification (SDA), rolling circle amplification (RCA), the loop-mediated isothermal amplification (LAMP), helicase-dependent amplification (HDA), as well as the recombinase polymerase amplification (RPA) or enzymatic recombinase amplification (ERA).

“Recombinase polymerase amplification” or “RPA” is a highly sensitive and selective isothermal amplification technique, operating at 37-42°° C. It has been used to amplify diverse targets, including RNA, miRNA, ssDNA and dsDNA from a wide variety of organisms and samples. “Enzymatic recombinase amplification” or “ERA” is another version of RPA with different thermally stable polymerase.

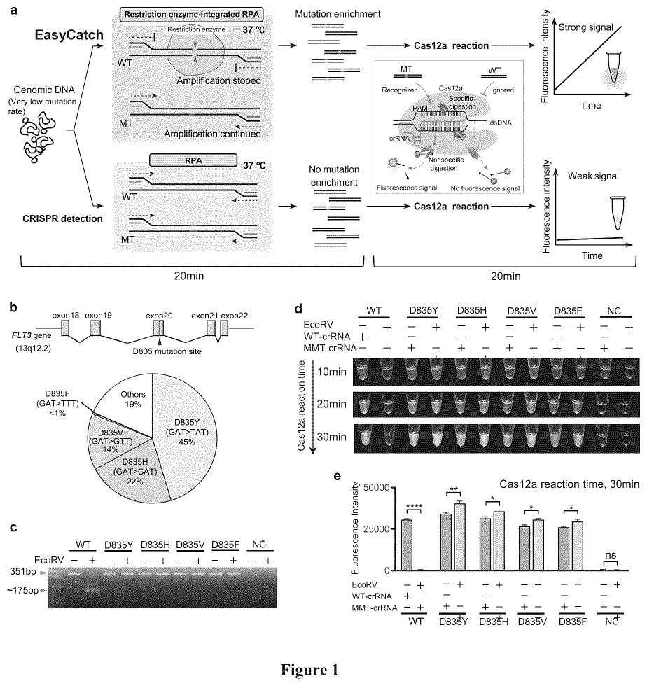

The RPA process starts when a recombinase protein (e.g., uvsX) from T4-like bacteriophages bind to primers in the presence of ATP and a crowding agent (a high molecular polyethyleneglycol), forming a recombinase-primer complex. The complex then interrogates double stranded DNA seeking a homologous sequence and promotes strand invasion by the primer at the cognate site. In order to prevent the ejection of the inserted primer by branch migration, the displaced DNA strand is stabilized by single-stranded binding proteins. Finally, the recombinase disassembles and a strand displacing DNA polymerase (e.g. large fragment of Bacillus subtilis Pol 1, Bsu) binds to the 3′ end of the primer to elongate it in the presence of dNTPs. Cyclic repetition of this process results in the achievement of exponential amplification (FIG. 1).

An “oligonucleotide primer” or “primer” refers to an oligonucleotide sequence that has a homologous sequence on a target nucleic acid and serves as a point of initiation of nucleic acid synthesis. Primers can be of a variety of lengths and are often less than 50 nucleotides in length, for example 12-30 nucleotides in length. The length and sequences of primers for use in nucleic acid amplification (e.g., PCR or RPA) can be designed based on principles known in the art.

The terms “nucleic acid” and “polynucleotide” are used interchangeably herein to refer to deoxyribonucleotides or ribonucleotides and polymers thereof in either single-or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide nucleic acids (PNAs).

The terms “about” and “approximately equal” are used herein to modify a numerical value and indicate a defined range around that value. If “X” is the value, “about X” or “approximately equal to X” generally indicates a value from 0.90X to 1.10X. Any reference to “about X” indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X.

Thus, “about X” is intended to disclose, e.g., “0.98X.” Thus, “from about 6 to 8.5” is equivalent to “from about 6 to about 8.5.” When “about” is applied to the first value of a set of values, it applies to all values in that set. Thus, “about 7, 9, or 11%” is equivalent to “about 7%, about 9%, or about 11%.”

### II. Introduction

Disclosed include methods, compositions, and kits for sensitively, accurately and efficiently amplifying, enriching and/or detecting a target nucleic acid, especially in a small amount (for example, less than 20%, less than 10%, less than 5%, less than 3%, less than 1%, less than 0.5% or less than 0.1% based on the total nucleic acids in a sample), in a sample by digesting or degrading non-target nucleic acids in the sample before or during amplification and may detect the amplified target nucleic acids. In one embodiment, the non-target nucleic acids in the sample, which generally are background nucleic acid molecules or wild type nucleic acid molecules without the target mutation(s) at specified site(s), are recognized by one or more proteins having an activity of cleaving nucleic acid at the specified site before or during amplification and cleaved. As a result, only the target nucleic acids with the alternation/mutation of interest are amplified and no or basically no non-target nucleic acids are amplified. Interference of the non-target nucleic acids during detection will be minimized and the sensitivity, accuracy and efficiency of the detection will be greatly increased. These methods, materials, and kits are especially suitable for convenient, sensitive and specific detection of rare targets (such as genetic variants and mutations, cancer-related mutations, etc.) for early cancer diagnosis and precision medicine.

### III. Method

Provided are methods for amplifying or enriching target nucleic acids with alternation(s) of interest at a specified site in a sample, including digesting or degrading non-target nucleic acids without the altemation(s) of interest at the specified site in the sample by exposing the non-target nucleic acids to one or more proteins having an activity of cleaving nucleic acid that recognizes the base(s) at the specified site before or during amplification.

Also provided are methods for detecting a target nucleic acid with altemation(s) of interest at a specified site in a sample, including digesting or degrading non-target nucleic acids without the alternation(s) of interest at the specified site in the sample by exposing the non-target nucleic acids to one or more proteins having an activity of cleaving nucleic acid that recognize the specified site before or during amplification, and detecting the amplified target nucleic acid.

In one or more embodiments, exposing the nucleic acids to the one or more proteins having an activity of cleaving nucleic acid is performed by adding the protein(s) in an amplification mixture for amplifying the target nucleic acid. In some embodiments, the amplification mixture is an isothermal nucleic acid amplification mixture.

As used herein, “alternation” and “mutation” refer to different base(s) at a specified site of the target nucleic acids as compared to the non-target nucleic acid sequence, including deletion, substitution and insertion of one or more base(s) at the specified site(s). Accordingly, the “non-target nucleic acid” herein refers to any nucleic acid without an alternation at a specified site that is needed to be digested to increase the amplification efficiency of the target nucleic acids. Generally, non-target nucleic acids are background nucleic acid molecules or wild type nucleic acid molecules without the target mutation(s) at specified site(s). Alternation may include alternation of two or more continuous bases as compared to the non-target nucleic acid. In some embodiments, alternation is a mutation of the target nucleic acid as compared to WT sequence. The alternation may include alternations known in the art, which cause diseases such as drug-induced deafness and congenital deafness, lead to severity of diseases or drug resistances, etc., including, HBV drug resistance mutation, tumor mutation, tumor or drug resistance mutation, tuberculosis drug resistance mutation, SARS-COV-2 mutation, FLT3-D835 mutation or the like. Examples of alternations or mutations include those summarized in Table 1.

In the present disclosure, the protein having an activity of cleaving nucleic acid that cleave the nucleic acid by recognizing the specified site digests the nucleic acid sequence without the alternation of interest at the specified site and leaves behind variants (i.e., with an alteration of interest), to enrich the target nucleotide sequences with alternation of interest for further detection.

Examples of proteins having an activity of cleaving nucleic acid include restriction endonuclease, Cas enzyme, Ago enzyme, ZFN enzyme, TALEN enzyme, and functional complexes thereof.

Restriction endonuclease used in the present disclosure may be any restriction endonuclease known in the art, include but is not limited to BclI, BsaBI, AclWI, Bst4CI, Xmil, BlsI, PspFI, CviKI-1, CviJI, MscI, EcoP15I, BspACI, BseLI, BstKTI, PspN4I, BspLI, NlaIV, Bmil, MaiI, RsnAI, BspEI, HpaII, Mrol, Kpn2I, BcoDI, BstDEI, BpulOI, BtsIMutI, BasBI, BtsCI, NspI, Fail, EcoRV and Btsal.

As used herein, the Ago enzyme includes but is not limited to pfAgo, cbAgo, LrAgo, pfAgo-mut, Apol, pfAgo. TtAgo and MjAgo.

As used herein the Cas enzyme includes but is not limited to Cas 9, Cas 12, Cas 13 and Cas 14, especially includes but is not limited to SpCas9, SaCas9, HypaCas9, StlCas9, spCas9-NG, LbCas 12a, spCas9-mut, and ScCas9.

A functional complex formed by Cas or Ago with their respective partner, such as Cas enzyme/sgRNA complex, Ago/gDNA complex and Cas 12a/crRNA complex, can also be used as a protein having an activity of cleaving nucleic acid. Preferably, the Cas enzyme/sgRNA complex is a Cas9/sgRNA complex, more preferably, a spCas9/sgRNA complex. Preferably, the Ago/gDNA complex is a pfAgo/gDNA complex. Preferably, the Cas12a/crRNA complex is a LbCas 12a/crRNA complex.

In the subject application, the terms sgRNA, gDNA and crRNA have a meaning commonly acknowledged in the art. In some embodiments, the protein having an activity of cleaving nucleic acid is a Cas enzyme, and the digesting or degrading step requires a gRNA (guide RNA) which forms a Cas enzyme/gRNA complex. That is, the digesting or degrading step is CRISPR-based digestion. In these embodiments, the guide RNA has a targeting region which binds to the non-target sequence to direct Cas enzyme cleavage of the bound sequence at a specified site. Guide RNAs can be designed based on principles known in the art. In preferable embodiments, guide RNA is designed to recognize WT FLT3 D835 sequence (-GATATC-) and the Cas enzyme/gRNA complex digest such sequence.

The protein having an activity of cleaving nucleic acid can cleave the nucleic acid at the specified site or other sites, depending on the particular protein used. Therefore, the recognizing site (i.e., the specified site) and the cleaving site can be the same or different in the non-target nucleic acid.

As a result of digestion by the proteins having an activity of cleaving nucleic acid, the non-target sequences are cleaved or degraded and amplification thereof are stopped before the cleavage site.

Amplification may be performed with a conventional amplification method, including helicase-dependent amplification (HAD), polymerase chain reaction (PCR), DNA ligase chain reaction (LCR), isothermal DNA amplification, QBeta RNA replicase, RNA transcription-based amplification reactions, loop-mediated isothermal amplification (LAMP), RT-LAMP, recombinase polymerase amplification (RPA), reverse transcription-recombinase polymerase amplification (RT-RPA), helicase-dependent amplification (HDA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), and isothermal multiple displacement amplification (IMDA).

Polymerase used in amplification may be any known polymerases and may be selected according to the used amplification method. Suitable DNA polymerases include but are not limited to DNA polymerases isolated or derived from Pyrococcus furiosus, Thermococcus litoralis, and Thermotoga maritime, Bacteriophage T4 or modified versions thereof.

In one or more preferred embodiments, a recombinase polymerase amplification (RPA) is used in the method of the subject application. In one embodiment, RPA are carried out in the presence of the protein(s) having an activity of cleaving nucleic acid. As such, cleavage of the non-target sequence and amplification of the target sequence are carried out sequentially or simultaneously, preferably in the same reaction system. As such, in a preferred embodiment, provided is a modified RPA method, which includes amplification of target nucleic acids with alternation of interest in the presence of the protein(s) having an activity of cleaving nucleic acid as described herein with which the non-target nucleic acids are digested or degraded before or during amplification.

Primers used in the amplification may be designed according to the sequence of the target nucleotide molecule or segment. This is well known in the art. Generally, the two primers of a primer pair are located on each side (i.e., downstream and upstream, respectively) of the site to be cleaved in the non-target nucleic acid. The cleaved sequence (e.g. wild-type sequence) cannot be amplified by the primer pair.

To detect the target nucleic acid, any suitable detection technique may be used, such as, DNA staining, nucleic acid amplification, spectrophotometry, sequencing, fluorescent probe hybridization, fluorescence resonance energy transfer, optical microscopy, electron microscopy, CRISPR-based detection, visual based detection, sensor-based detection, color detection, gold nanoparticle based detection, electrochemical detection, semiconductor-based sensing, or combinations thereof. Sequencing may be FGS or NGS. Nucleic acid amplification may be qPCR.

In one or more embodiments, the target nucleic acid is detected with one or more protein(s) capable of recognizing a specific nucleic acid sequence, or a functional complex thereof. The specific nucleic acid sequence generally includes the mutation site.

Protein(s) capable of recognizing a specific nucleic acid sequence, i.e., protein(s) for detection, include but is not limited to Cas enzyme, Ago enzyme, ZFN enzyme, TALEN enzyme, and functional complexes thereof. In some embodiments, the detection is a CRISPR-based detection based on any known Cas proteins

As used herein the Cas enzyme includes but is not limited to Cas 9, Cas 12, Cas 13 and Cas 14, especially includes but is not limited to SpCas9, SaCas9, HypaCas9, StlCas9, spCas9-NG, LbCas12a, spCas9-mut, and ScCas9.

As used herein, the Ago enzyme includes but is not limited to pfAgo, cbAgo, LrAgo, pfAgo-mut, Apol, pfAgo, TtAgo and MjAgo.

A functional complex formed by Cas or Ago with their respective partner, such as Cas enzyme/sgRNA complex, Ago/gDNA complex and Cas 12a/crRNA complex, can also be used as a protein for detection. Preferably, the Cas enzyme/sgRNA complex is a Cas9/sgRNA complex, more preferably, a spCas9/sgRNA complex. Preferably, the Ago/gDNA complex is a pfAgo/gDNA complex. Preferably, the Cas 12a/crRNA complex is a LbCas12a/crRNA complex.

In some embodiments, the protein capable of recognizing a specific nucleic acid sequence include but is not limited to LbCas 12a, FnCas12a, Lb5Cas12a, HkCas12a, TsCas 12a, BbCas12a, BoCas 12a, Lb4Cas 12a, LbuCas13a, LwaCas13a, LbaCas13a, PprCas13a, HheCas13a, EreCas13a, AsCas12a, TsCas12a, BbCas12a, BoCas 12a, Lb4Cas 12a, spCas9, pfAgo, cbAgo, LrAgo, Cas12b, Cas12a-mut, Cas 12b-mut, AapCas12b, BrCas 12b, CcaCas13b, PsmCas13b and AacCas12b, or functional complexes thereof.

Cleavage of the non-target sequence, amplification of the target sequence and the detection of the target sequence may be carried out sequentially or simultaneously, preferably in the same reaction system.

The methods of the present disclosure may be used for the detection of clinically actionable information about a subject or a tumor in a patient, to detect and describe mutations and/or alterations in DNA of hematologic cancer cell in a blood or plasma sample that also contains an abundance of “normal”, somatic DNA, to monitor cancer remission, to inform treatment, such as dosage regime or immunotherapy treatment, to be used with fetal DNA to detect, for example, mutations characteristic of inherited genetic disorders, to detect and describe mutations and/or alterations in circulating tumor DNA in a blood or plasma sample that also contains an abundance of “normal”, somatic DNA. The DNA may include circulating tumor DNA in a patient's blood or plasma, or fetal DNA in maternal blood or plasma.

The term “hematologic cancer” is a group of malignant diseases that arise from cells in the bone marrow or lymphatic tissues, including but not limited to leukemia, lymphoma and myeloma, such as acute lymphocytic leukemia (ALL).

The methods of the present disclosure may include detection or isolation of hematologic cancer cells from a blood sample. The methods of the present disclosure may include detection or isolation of lymphocyte (e.g., PBMC, WBC) from a blood sample of a subject suffering hematologic cancer. For example, to isolating WBCs, red cells in peripheral blood or bone marrow blood samples are lysed, and unlysed WBCs is separated from lysed RBCs simply by centrifuge. Genomic DNA can be extracted by nucleic acid releaser (e.g., Suzhou GenDx Biotech, China).

The methods of the present disclosure may include detection or isolation of circulating tumor cells (CTCs) from a blood sample. CTC The methods of the present disclosure may employ an enrichment step to optimize the probability of rare cell detection, achievable through immune-magnetic separation, centrifugation or filtration.

The methods of the present disclosure can be used to detecting a target RNA, which may include reverse transcription from RNA to DNA. Such method may further include isolation of RNA from a sample (such as virus). Means for isolation of RNA and/or reverse transcription of RNA are well known in the art.

When a genomic alteration is thus detected, a report may be provided to, for example, describe the alteration in a patient.

Knowledge of a mutational landscape of a tumor may be used to inform treatment decisions, monitor therapy, detect remissions, or combinations thereof. For example, where the report includes a description of multiple mutations, the report may also include an estimate of a tumor mutation burden (TMB) for a tumor. It may be found that TMB is predictive of success of immunotherapy in treating a tumor, and thus the methods described herein may be used for treating a tumor.

Examples of target nucleic acids and their respective mutations are listed in No. 1-85 as shown in Table 1. Proteins for cleavage and amplification method for purpose of amplification or enrichment and proteins for detection, if necessary, are also listed for each of target nucleic acids. It should be understood that the protein for cleavage, the amplification method and the protein for detection listed for each of target nucleic acids is not the sole protein for cleavage, amplification method and protein for detection for amplifying, enriching and detecting that target nucleic acid. It can be determined that a suitable protein for cleavage, a suitable amplification method and/or a suitable protein for detection for each of the target nucleic acids with the present disclosure and the prior art.

TABLE 1

| | | | Proteins for | Proteins for | amplification | |
|---|---|---|---|---|---|---|
| No. | target name | mutation | cleavage | detection | method | note |
| 1 | 12S rRNA 1494C > T | 1494C > T | spCas9 | LbCas12a | RT-RPA | Drug-induced deafness |
| 2 | 12S rRNA 1555A > G | 1555A > G | spCas9 | LbCas12a | RT-LAMP | HBV drug resistance mutation |
| 3 | ALK G1202R | c.3604G > A | spCas9 | FnCas12a | RPA | Tumor mutation |
| 4 | ALK I1171T | c.3512T > C | BclI | Lb5Cas12a | LAMP | Tumor mutation |
| 5 | ALK L1196M | c.3586C > A | pfAgo | HkCas12a | HAD | Congenital deafness |
| 6 | BRAF G466V | c.1397G > T | BsaBI | TsCas12a | SDA | Tumor or drug resistance |
| | | | | | | mutation |
| 7 | BRAF G469R | c.1405G > A | AclWI | BbCas12a | NASBA | Tumor or drug resistance |
| | | | | | | mutation |
| 8 | BRAF G469V | c.1406G > T | Cas | BoCas12a | TMA | Tumor or drug resistance |
| | | | | | | mutation |
| 9 | BRAF V600D | c.1799_1800delinsAC | Bst4CI | Lb4Cas12a | NEAR | Tumor or drug resistance |
| | | | | | | mutation |
| 10 | BRAF V600D | c.1799_1800delinsAT | cbAgo | LbuCas13a | RCA | Tumor or drug resistance |
| | | | | | | mutation |
| 11 | BRAF V600E | c.1799T > A | LrAgo | LwaCas13a | MDA | Tumor or drug resistance |
| | | | | | | mutation |
| 12 | BRAF V600E | c.1799_1800delinsAA | SaCas9 | LbaCas13a | RAM | Tumor or drug resistance |
| | | | | | | mutation |
| 13 | BRAF V600K | c.1798_1799delinsAA | HypaCas9 | PprCas13a | cHDA | Tumor or drug resistance |
| | | | | | | mutation |
| 14 | BRAF V600R | c.1798_1799delinsAG | St1Cas9 | HheCas13a | SMART | Tumor or drug resistance |
| | | | | | | mutation |
| 15 | BRAF Y472C | c.1415A > G | XmiI | EreCas13a | 3SR | Tumor or drug resistance |
| | | | | | | mutation |
| 16 | BTK C481S | c.G > A | BlsI | AsCas12a | NEAR | Tumor or drug resistance |
| | | | | | | mutation |
| 17 | EGFR G719A | c.2156G > C | PspFI | LbCas12a | RPA | Tumor or drug resistance |
| | | | | | | mutation |
| 18 | EGFR G719C | c.2155G > T | CviKI-1 | LbCas12a | LAMP | Tumor or drug resistance |
| | | | | | | mutation |
| 19 | EGFR G719S | c.2155G > A | CviJI | AsCas12a | HAD | Tumor or drug resistance |
| | | | | | | mutation |
| 20 | EGFR L858R | c.2573T > G | MscI | TsCas12a | SDA | Tumor or drug resistance |
| | | | | | | mutation |
| 21 | EGFR L861Q | c.2582T > A | spCas9 | BbCas12a | NASBA | Tumor or drug resistance |
| | | | | | | mutation |
| 22 | EGFR S768I | c.2303G > T | EcoP15I | BoCas12a | TMA | Tumor or drug resistance |
| | | | | | | mutation |
| 23 | EGFR T790M | c.2369C > T | spCas9-NG | Lb4Cas12a | LAMP | Tumor or drug resistance |
| | | | | | | mutation |
| 24 | ERBB2 G660R | c.1978G > C | BspACI | LbuCas13a | GEAR | Tumor or drug resistance |
| | | | | | | mutation |
| 25 | ERBB2 L755S | c.2264T > C | BseLI | LwaCas13a | IMDA | Tumor or drug resistance |
| | | | | | | mutation |
| 26 | ERBB2 R678Q | c.2033G > A | Lbcas12a | LbaCas13a | PCR | Tumor or drug resistance |
| | | | | | | mutation |
| 27 | ERBB2 S310F | c.929C > T | BstKTI | spCas9 | 3SR | Tumor or drug resistance |
| | | | | | | mutation |
| 28 | ERBB2 S310Y | c.929C > A | PspN4I | pfAgo | NEAR | Tumor or drug resistance |
| | | | | | | mutation |
| 29 | ERBB2 S310Y | c.929_930delinsAT | BspLI | cbAgo | RPA | Tumor or drug resistance |
| | | | | | | mutation |
| 30 | ERBB2 S310A | c.928T > G | NlaIV | LrAgo | LAMP | Tumor or drug resistance |
| | | | | | | mutation |
| 31 | ERBB2 S310* | c.930C > A | BmiI | Cas12b | HAD | Tumor or drug resistance |
| | | | | | | mutation |
| 32 | ERBB2 V659E | c.1976_1977delinsAG | MaiI | Cas12a-mut | SDA | Tumor or drug resistance |
| | | | | | | mutation |
| 33 | ERBB2 V659D | c.1976T > A | spCas9-mut | AapCas12b | NASBA | Tumor or drug resistance |
| | | | | | | mutation |
| 34 | ERBB2 V659L | c.1975G > C | pfAgo-mut | BrCas12b | TMA | Tumor or drug resistance |
| | | | | | | mutation |
| 35 | ERBB2 V842I | c.2524G > A | RsnAI | AacCas12b | LAMP | Tumor or drug resistance |
| | | | | | | mutation |
| 36 | ALK F1174L | c.3522C > A | Cas12 | FnCas12a | GEAR | Tumor or drug resistance |
| | | | | | | mutation |
| 37 | ALK F1174L | c.3520T > C | ApoI | Lb5Cas12a | IMDA | Tumor or drug resistance |
| | | | | | | mutation |
| 38 | ALK F1174L | c.3522C > G | Cas14 | HkCas12a | PCR | Tumor or drug resistance |
| | | | | | | mutation |
| 39 | ALK L1196M | c.3586C > A | Cas9 | TsCas12a | RPA | Tumor or drug resistance |
| | | | | | | mutation |
| 40 | GJB2 176delC | 176delC | spCas9 | BbCas12a | LAMP | congenital deafness |
| 41 | GJB2 35delG | 35delG | pfAgo | BoCas12a | HAD | congenital deafness |
| 42 | IDH1 R132H | c.395G > A | TtAgo | Lb4Cas12a | SDA | Tumor or drug resistance |
| | | | | | | mutation |
| 43 | IDH2 R140Q | c.419G > A | BspEI | LbuCas13a | NASBA | Tumor or drug resistance |
| | | | | | | mutation |
| 44 | IDH2 R140L | c.419G > T | HpaII | LwaCas13a | TMA | Tumor or drug resistance |
| | | | | | | mutation |
| 45 | IDH2 R140W | c.418C > T | MroI | LbaCas13a | LAMP | Tumor or drug resistance |
| | | | | | | mutation |
| 46 | IDH2 R140G | c.418C > G | Kpn2I | PprCas13a | GEAR | Tumor or drug resistance |
| | | | | | | mutation |
| 47 | IDH2 R172K | c.515G > A | MjAgo | HheCas13a | IMDA | Tumor or drug resistance |
| | | | | | | mutation |
| 48 | JAK2 V617F | c.1849G > T | spCas9 | EreCas13a | PCR | Tumor or drug resistance |
| | | | | | | mutation |
| 49 | JAK2 V617F | c.1848_1849delinsCT | ZFN | AsCas12a | 3SR | Tumor or drug resistance |
| | | | | | | mutation |
| 50 | katG315 | AGC > ACC | scCas9 | LbCas12a | NEAR | Tuberculosis drug |
| | | | | | | resistance mutation |
| 51 | KIT D816V | c.2447A > T | BcoDI | LbCas12a | RPA | Tumor or drug resistance |
| | | | | | | mutation |
| 52 | KRAS G12A | c.35G > C | spCas9 | AsCas12a | LAMP | Tumor or drug resistance |
| | | | | | | mutation |
| 53 | KRAS G12C | c.34G > T | spCas9-NG | TsCas12a | HAD | Tumor or drug resistance |
| | | | | | | mutation |
| 54 | KRAS G12D | c.35G > A | spCas9-NG | BbCas12a | SDA | Tumor or drug resistance |
| | | | | | | mutation |
| 55 | KRAS G12R | c.34G > C | spCas9-NG | BoCas12a | NASBA | Tumor or drug resistance |
| | | | | | | mutation |
| 56 | KRAS G12S | c.34G > A | spCas9-NG | Lb4Cas12a | TMA | Tumor or drug resistance |
| | | | | | | mutation |
| 57 | KRAS G12V | c.35G > T | spCas9-NG | LbuCas13a | LAMP | Tumor or drug resistance |
| | | | | | | mutation |
| 58 | KRAS G13C | c.37G > T | spCas9-NG | LwaCas13a | PCR | Tumor or drug resistance |
| | | | | | | mutation |
| 59 | KRAS G13D | c.38G > A | spCas9-NG | LbaCas13a | RPA | Tumor or drug resistance |
| | | | | | | mutation |
| 60 | KRAS G13S | c.37G > A | spCas9-NG | spCas9 | LAMP | Tumor or drug resistance |
| | | | | | | mutation |
| 61 | MET D1228H | G > C | saCas9 | pfAgo | HAD | Tumor or drug resistance |
| | | | | | | mutation |
| 62 | MET H1106D | C > G | scCas9 | cbAgo | SDA | Tumor or drug resistance |
| | | | | | | mutation |
| 63 | MET V1092I | G > A | spCas9-NG | LrAgo | NASBA | Tumor or drug resistance |
| | | | | | | mutation |
| 64 | MET V1188L | G > C | TALEN | Cas12b | TMA | Tumor or drug resistance |
| | | | | | | mutation |
| 65 | MET Y1230C | A > G | ZFN | Cas12a-mut | LAMP | Tumor or drug resistance |
| | | | | | | mutation |
| 66 | MET Y1230H | T > C | Ago | AapCas12b | GEAR | Tumor or drug resistance |
| | | | | | | mutation |
| 67 | MPL S505 | c.1513A > T | BstDEI | BrCas12b | IMDA | Tumor or drug resistance |
| | | | | | | mutation |
| 68 | MPL W515 | c.1543T > A | Bpu10I | AacCas12b | PCR | Tumor or drug resistance |
| | | | | | | mutation |
| 69 | NRAS Q61H | c.183A > C | spCas9 | AacCas12b | RPA | Tumor or drug resistance |
| | | | | | | mutation |
| 70 | NRAS Q61K | c.181C > A | spCas9 | FnCas12a | LAMP | Tumor or drug resistance |
| | | | | | | mutation |
| 71 | NRAS Q61L | c.182A > T | spCas9 | Lb5Cas12a | RPA | Tumor or drug resistance |
| | | | | | | mutation |
| 72 | NRAS Q61R | c.182A > G | spCas9 | HkCas12a | LAMP | Tumor or drug resistance |
| | | | | | | mutation |
| 73 | PIK3CA E545K | c.1633G > A | BtsIMutI | TsCas12a | PCR | Tumor or drug resistance |
| | | | | | | mutation |
| 74 | PIK3CA H1047R | c.3140A > G | BasBI | BbCas12a | RPA | Tumor or drug resistance |
| | | | | | | mutation |
| 75 | MTB rpoBD516V | 516 A > T | spCas9 | BoCas12a | LAMP | Tuberculosis drug |
| | | | | | | resistance mutation |
| 76 | MTB katG N315M | 315 AGC → ACA | SaCas9 | LbCas12a | RPA | Tuberculosis drug |
| | | | | | | resistance mutation |
| 77 | HBV rtM204I | T > G | spCas9 | LwaCas13a | LAMP | HBV drug resistance |
| | | | | | | mutation |
| 78 | HBV rtM204V | T > C | scCas9 | LbaCas13a | | HBV drug resistance |
| | | | | | | mutation |
| 79 | SLC26A4 2168A > G | 2168A > G | spCas9 | HheCas13a | TMA | Drug-induced deafness |
| 80 | STAT3 D661V | c.1982A > T | BtsCI | EreCas13a | LAMP | Tumor or drug resistance |
| | | | | | | mutation |
| 81 | STAT3 N647I | c.1940A > T | NspI | AsCas12a | GEAR | Tumor or drug resistance |
| | | | | | | mutation |
| 82 | STAT3 Y640F | c.1919A > T | FaiI | LbCas12a | IMDA | Tumor or drug resistance |
| | | | | | | mutation |
| 83 | TERT C228T | c.865A > T | BtsaI | LbCas12a | RPA | Tumor or drug resistance |
| | | | | | | mutation |
| 84 | SARS-CoV-2 | GAA > CAA | pfAgo | AsCas12a | RT-LAMP | SARS-CoV-2 mutation |
| | E484Q | | | | | |
| 85 | FLT3 D835 | GAT > TAT | EcoRV | LbCas12a | RPA | FLT3-D835 mutation |

### IV. Kit

Also provided is a kit for amplifying or enriching and may detect target nucleic acids with alternation(s) of interest at a specified site in a sample, including reagent(s) for amplification of target nucleic acids with alternation(s) of interest at the specified site in the sample, and reagent(s) for digesting non-target nucleic acids without the alternation(s) of interest at the specified site in the sample.

The reagent(s) for amplification of target nucleic acids with alternation(s) of interest at the specified site in the sample may be any one or more reagents used in any known amplification methods, including but is not limited to helicase-dependent amplification (HAD), polymerase chain reaction (PCR), DNA ligase chain reaction (LCR), isothermal DNA amplification, QBeta RNA replicase, RNA transcription-based amplification reactions, loop-mediated isothermal amplification (LAMP), RT-LAMP, recombinase polymerase amplification (RPA), reverse transcription-recombinase polymerase amplification (RT-RPA), helicase-dependent amplification (HDA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), and isothermal multiple displacement amplification (IMDA).

In some preferred embodiments, the reagent(s) for amplification include one or more reagent(s) for PCR or isothermal amplification reaction.

Examples of the reagent(s) for amplification include one or more of reaction buffer, polymerase (thermally stable polymerase or isothermal polymerase), primers, dNTP, activator, ddH₂O, or single stranded DNA binding (SSB). The buffer can contain one or more buffer components and salts. In some embodiments, the buffer component is Tris-HCl. In some embodiments, the salts are KCI and MgCl₂. Isothermal amplification system includes the GenDx ERA Kit sold by Suzhou GenDx Biotech, China. As described herein, the two primers of a primer pair are located on each side (i.e., downstream and upstream, respectively) of the site to be cleaved in the non-target nucleic acid. As such, the cleaved sequence (e.g. wild-type sequence) cannot be amplified by the primer pair. Polymerase may be any known polymerase and may be selected according to the used amplification method. Suitable DNA polymerases include but are not limited to DNA polymerases isolated or derived from Pyrococcus furiosus, Thermococcus litoralis, and Thermotoga maritime, Bacteriophage T4 or modified versions thereof.

In some preferred embodiments, the reagents for amplification include reagents used for RPA.

The reagent(s) for digesting non-target nucleic acids without the alternation(s) of interest at the specified site in the sample includes the protein having an activity of cleaving nucleic acid as described herein.

Examples of proteins having an activity of cleaving nucleic acid include restriction endonuclease, Cas enzyme, Ago enzyme, ZFN enzyme, TALEN enzyme, and functional complexes thereof.

Restriction endonuclease used in the present disclosure may be any restriction endonuclease known in the art, include but is not limited to BclI, BsaBI, AclWI, Bst4CI, Xmil, BlsI, PspFI, CviKI-1, CviJI, MscI, EcoP15I, BspACI, BseLI, BstKTI, PspN4I, BspLI, NlaIV, Bmil, MaiI, RsnAI, BspEI, HpaII, Mrol, Kpn21, BcoDI, BstDEI, BpulOI, BtsIMutI, BasBI, BtsCI, NspI, Fail, EcoRV and Btsal.

The Ago enzyme includes but is not limited to pfAgo, cbAgo, LrAgo, pfAgo-mut, Apol, pfAgo, TtAgo and MjAgo.

The Cas enzyme includes but is not limited to Cas 9, Cas 12, Cas 13 and Cas 14, especially includes but is not limited to SpCas9, SaCas9, HypaCas9, StlCas9, spCas9-NG, LbCas12a, spCas9-mut, and ScCas9.

A functional complex formed by Cas or Ago with their respective partner, such as Cas enzyme/sgRNA complex, Ago/gDNA complex and Cas 12a/crRNA complex, can also be used as a protein having an activity of cleaving nucleic acid. Preferably, the Cas enzyme/sgRNA complex is a Cas9/sgRNA complex, more preferably, a spCas9/sgRNA complex. Preferably, the Ago/gDNA complex is a pfAgo/gDNA complex. Preferably, the Cas12a/crRNA complex is a LbCas 12a/crRNA complex.

The kit can further include reagent(s) used for detecting the target nucleic acid. The reagent(s) used for detecting the target nucleic acids include one or more reagents used for DNA staining, nucleic acid amplification, spectrophotometry, sequencing, fluorescent probe hybridization, fluorescence resonance energy transfer, optical microscopy, electron microscopy, CRISPR-based detection, or combinations thereof.

In one or more embodiments, the reagent(s) used for detecting the target nucleic acids include one or more protein(s) capable of recognizing a specific nucleic acid sequence, or a functional complex thereof.

The protein(s) capable of recognizing a specific nucleic acid sequence, i.e., protein(s) for detection, include but is not limited to Cas enzyme, Ago enzyme, ZFN enzyme, TALEN enzyme, and functional complexes thereof. In some embodiments, the detection is a CRISPR-based detection based on any known Cas proteins.

The Cas enzyme includes but is not limited to Cas 9, Cas 12, Cas 13 and Cas 14, especially includes but is not limited to SpCas9, SaCas9, HypaCas9, StlCas9, spCas9-NG, LbCas12a, spCas9-mut, and ScCas9.

The Ago enzyme includes but is not limited to pfAgo, cbAgo, LrAgo, pfAgo-mut, Apol, pfAgo, TtAgo and MjAgo.

A functional complex formed by Cas or Ago with their respective partner, such as Cas enzyme/sgRNA complex, Ago/gDNA complex and Cas 12a/crRNA complex, can also be used as a protein for detection. Preferably, the Cas enzyme/sgRNA complex is a Cas9/sgRNA complex, more preferably, a spCas9/sgRNA complex. Preferably, the Ago/gDNA complex is a pfAgo/gDNA complex. Preferably, the Cas 12a/crRNA complex is a LbCas12a/crRNA complex.

In some embodiments, the protein capable of recognizing a specific nucleic acid sequence include but is not limited to LbCas 12a, FnCas12a, Lb5Cas12a, HkCas12a, TsCas12a, BbCas12a, BoCas 12a, Lb4Cas 12a, LbuCas13a, LwaCas 13a, LbaCas13a, PprCas13a, HheCas13a, EreCas13a, AsCas12a, TsCas12a, BbCas12a, BoCas 12a, Lb4Cas12a, spCas9, pfAgo, cbAgo, LrAgo, Cas12b, Cas12a-mut, Cas 12b-mut, AapCas12b, BrCas 12b, CcaCas13b, PsmCas13b and AacCas12b, or functional complexes thereof.

Preferably, the kit contains a mixture including reagent(s) for RPA and the reagent(s) for digesting the non-target sequence, especially protein(s) having an activity of cleaving nucleic acid as described herein. In some embodiments, the mixture further includes reagent(s) used for segment detection.

Most preferably, the kit includes a protein for cleavage listed in any one of the ID No. in Table A and reagent(s) for the amplification method listed in the same ID No., for amplifying or enriching the target of the same ID No. In some other embodiments, the kit further includes the protein for detection listed in the same ID No. for detection of the target of the same ID No.

TABLE A

| | | | | amplification |
|---|---|---|---|---|
| ID No. | target name | Protein for cleavage | Protein for detection | method |
| 1 | 12S rRNA 1494C>T | spCas9 | LbCas12a | RT-RPA |
| 2 | 12S rRNA 1555A>G | spCas9 | LbCas12a | RT-LAMP |
| 3 | ALK G1202R | spCas9 | FnCas12a | RPA |
| 4 | ALK I1171T | BclI | Lb5Cas12a | LAMP |
| 5 | ALK L1196M | pfAgo | HkCas12a | HAD |
| 6 | BRAF G466V | BsaBI | TsCas12a | SDA |
| 7 | BRAF G469R | AclWI | BbCas12a | NASBA |
| 8 | BRAF G469V | Cas | BoCas12a | TMA |
| 9 | BRAF V600D | Bst4CI | Lb4Cas12a | NEAR |
| 10 | BRAF V600D | cbAgo | LbuCas13a | RCA |
| 11 | BRAF V600E | LrAgo | LwaCas13a | MDA |
| 12 | BRAF V600E | SaCas9 | LbaCas13a | RAM |
| 13 | BRAF V600K | HypaCas9 | PprCas13a | cHDA |
| 14 | BRAF V600R | St1Cas9 | HheCas13a | SMART |
| 15 | BRAF Y472C | XmiI | EreCas13a | 3SR |
| 16 | BTK C481S | BlsI | AsCas12a | NEAR |
| 17 | EGFR G719A | PspFI | LbCas12a | RPA |
| 18 | EGFR G719C | CviKI-1 | LbCas12a | LAMP |
| 19 | EGFR G719S | CviJI | AsCas12a | HAD |
| 20 | EGFR L858R | MscI | TsCas12a | SDA |
| 21 | EGFR L861Q | spCas9 | BbCas12a | NASBA |
| 22 | EGFR S768I | EcoP15I | BoCas12a | TMA |
| 23 | EGFR T790M | spCas9-NG | Lb4Cas12a | LAMP |
| 24 | ERBB2 G660R | BspACI | LbuCas13a | GEAR |
| 25 | ERBB2 L755S | BseLI | LwaCas13a | IMDA |
| 26 | ERBB2 R678Q | Lbcas12a | LbaCas13a | PCR |
| 27 | ERBB2 S310F | BstKTI | spCas9 | 3SR |
| 28 | ERBB2 S310Y | PspN4I | pfAgo | NEAR |
| 29 | ERBB2 S310Y | BspLI | cbAgo | RPA |
| 30 | ERBB2 S310A | NlaIV | LrAgo | LAMP |
| 31 | ERBB2 S310* | BmiI | Cas12b | HAD |
| 32 | ERBB2 V659E | MaiI | Cas12a-mut | SDA |
| 33 | ERBB2 V659D | spCas9-mut | AapCas12b | NASBA |
| 34 | ERBB2 V659L | pfAgo-mut | BrCas12b | TMA |
| 35 | ERBB2 V842I | RsnAI | AacCas12b | LAMP |
| 36 | ALK F1174L | Cas12 | FnCas12a | GEAR |
| 37 | ALK F1174L | ApoI | Lb5Cas12a | IMDA |
| 38 | ALK F1174L | Cas14 | HkCas12a | PCR |
| 39 | ALK L1196M | Cas9 | TsCas12a | RPA |
| 40 | GJB2 176delC | spCas9 | BbCas12a | LAMP |
| 41 | GJB2 35delG | pfAgo | BoCas12a | HAD |
| 42 | IDH1 R132H | TtAgo | Lb4Cas12a | SDA |
| 43 | IDH2 R140Q | BspEI | LbuCas13a | NASBA |
| 44 | IDH2 R140L | HpaII | LwaCas13a | TMA |
| 45 | IDH2 R140W | MroI | LbaCas13a | LAMP |
| 46 | IDH2 R140G | Kpn2I | PprCas13a | GEAR |
| 47 | IDH2 R172K | MjAgo | HheCas13a | IMDA |
| 48 | JAK2 V617F | spCas9 | EreCas13a | PCR |
| 49 | JAK2 V617F | ZFN | AsCas12a | 3SR |
| 50 | katG315 | scCas9 | LbCas12a | NEAR |
| 51 | KIT D816V | BcoDI | LbCas12a | RPA |
| 52 | KRAS G12A | spCas9 | AsCas12a | LAMP |
| 53 | KRAS G12C | spCas9-NG | TsCas12a | HAD |
| 54 | KRAS G12D | spCas9-NG | BbCas12a | SDA |
| 55 | KRAS G12R | spCas9-NG | BoCas12a | NASBA |
| 56 | KRAS G12S | spCas9-NG | Lb4Cas12a | TMA |
| 57 | KRAS G12V | spCas9-NG | LbuCas13a | LAMP |
| 58 | KRAS G13C | spCas9-NG | LwaCas13a | PCR |
| 59 | KRAS G13D | spCas9-NG | LbaCas13a | RPA |
| 60 | KRAS G13S | spCas9-NG | spCas9 | LAMP |
| 61 | MET D1228H | saCas9 | pfAgo | HAD |
| 62 | MET H1106D | scCas9 | cbAgo | SDA |
| 63 | MET V1092I | spCas9-NG | LrAgo | NASBA |
| 64 | MET V1188L | TALEN | Cas12b | TMA |
| 65 | MET Y1230C | ZFN | Cas12a-mut | LAMP |
| 66 | MET Y1230H | Ago | AapCas12b | GEAR |
| 67 | MPL S505 | BstDEI | BrCas12b | IMDA |
| 68 | MPL W515 | Bpu10I | AacCas12b | PCR |
| 69 | NRAS Q61H | spCas9 | AacCas12b | RPA |
| 70 | NRAS Q61K | spCas9 | FnCas12a | LAMP |
| 71 | NRAS Q61L | spCas9 | Lb5Cas12a | RPA |
| 72 | NRAS Q61R | spCas9 | HkCas12a | LAMP |
| 73 | PIK3CA E545K | BtsIMutI | TsCas12a | PCR |
| 74 | PIK3CA H1047R | BasBI | BbCas12a | RPA |
| 75 | MTB rpoBD516V | spCas9 | BoCas12a | LAMP |
| 76 | MTB katG N315M | SaCas9 | LbCas12a | RPA |
| 77 | HBV rtM204I | spCas9 | LwaCas13a | LAMP |
| 78 | HBV rtM204V | scCas9 | LbaCas13a | RPA |
| 79 | SLC26A4 2168A>G | spCas9 | HheCas13a | TMA |
| 80 | STAT3 D661V | BtsCI | EreCas13a | LAMP |
| 81 | STAT3 N647I | NspI | AsCas12a | GEAR |
| 82 | STAT3 Y640F | FaiI | LbCas12a | IMDA |
| 83 | TERT C228T | BtsaI | LbCas12a | RPA |
| 84 | SARS-CoV-2 E484Q | pfAgo | AsCas12a | RT-LAMP |
| 85 | FLT3 D835 | EcoRV | LbCas12a | RPA |

For example, the kit including the protein for cleavage and reagent(s) for the amplification method listed in ID No. 1 may include spCas9 and reagent(s) for performing RT-RPA, and is used to amplify or enrich a fragment of 12S rRNA containing 1494C>T. The kit may further includes a protein for detection which is LbCas12a for detection of the fragment.

5 The kit may also include instructions or other materials such as pre-formatted report shells that receive information from the methods to provide a report. The reagents, instructions, and any other useful materials may be packaged in a suitable container. Kits of the disclosure may be made to order. For example, an investigator may use, e.g., an online tool to design guide RNA and reagents for the performance of methods herein. The guide RNAs may be synthesized using a suitable synthesis instrument. The synthesis instrument may be used to synthesize 10 oligonucleotides such as gRNAs or single-guide RNAs (sgRNAs). Any suitable instrument or chemistry may be used to synthesize a gRNA. The resultant reagents (e.g., guide RNAs, and endonuclease(s)) can be packaged in a container for shipping as a kit.

In present disclosure, restriction digestion was included in the amplification (e.g., Recombinase Polymerase Amplification (RPA)) step to destroy nucleic acids without an alteration or certain alteration (e.g., wild-type sequences), thus presumably enhancing detection sensitivity. Using FLT3 D835 mutations as a model, the inventor optimized the method by crRNAs and RPA primers screening, and compared it with conventional methods (FGS, NGS, and qPCR) on a series of plasmid templates and 112 clinical samples. A method herein reached a sensitivity of 0.001% for detecting FLT3 D835 mutations, which represented the highest level achieved by any mutation detection methods. The entire workflow (from sample preparation to data output) took only an hour and required only simple instruments and operations. Similar detection sensitivity and accuracy were also achieved for all the other cancer mutations, i.e., IDH2 R172K. EGFR L858R and e19del, and NRAS G12D, showing the method will be invaluable for point-of-care cancer diagnosis and precision medicine.

The present disclosure further provides use of one or more protein(s) having an activity of cleaving nucleic acid that recognizes base(s) at a specified site of non-target nucleic acids in the manufacture of a reagent or a kit for amplifying or enriching target nucleic acids with alternation(s) of interest at the specified site as compared to the target nucleic acids. Preferably, in addition to the protein(s), the reagent or the kit may further include reagents for performing amplification of the target nucleic acids, such as primers, buffer or the like. The amplification may be any one of the amplifications as disclosed herein.

Also provided is use of one or more protein(s) having an activity of cleaving nucleic acid that recognizes base(s) at a specified site of non-target nucleic acids in the manufacture of a reagent or a kit for detecting target nucleic acids with alternation(s) of interest at the specified site as compared to the target nucleic acids. Preferably, in addition to the protein(s), the reagent or the kit may further include reagents for performing amplification of the target nucleic acids, such as primers, buffer or the like, and/or reagents for detecting the amplified target nucleic acids. The amplification may be any one of the amplifications as disclosed herein, and the detection may be any one of the detection as disclosed herein.

### EXAMPLES

The following example is provided in order to better enable one of ordinary skill in the art to make and use the disclosed compositions and methods, and is not intended to limit the scope of the disclosure in any way.

### Methods

### Samples Treatment

A total of 112 AML patient samples were collected from the Hematology Department in Zhongnan Hospital of Wuhan University under an approved Institutional Review Board protocol. For genomic DNA extraction, 200˜500 μl peripheral blood or bone marrow blood samples were mixed upside down four times with the red cell lysing reagent (Biosharp, Hefei, China). Then, 1 minute of minicentrifuge was used to separate lysed RBCs and unlysed WBCs. Finally, the precipitated WBCs were split by a 100 μl nucleic acid releaser (Suzhou GenDx Biotech, China) at 95° C. for 3 min to release genomic DNA. Two microliters of the treated sample were used for the subsequent assay.

### Plasmid Construction

A 675-bp DNA fragment covering the WT FLT3-D835 site was PCR amplified with primers P1 and P2 from a wild-type patient's genomic DNA, then fused and cloned into the pGem-T vector (Takara, China). After that, the recombinant plasmids were transformed into E. coli DH5α, extracted with an Axy Prep Plasmid Miniprep Kit (Axygen, CA, USA), and quantified by a Nanodrop2000 (Thermo Fisher Scientific, MA, USA). For the construction of FLT3-D835Y/H/V/F plasmids, primers P3 and P4 carrying the D835Y mutation, primers P5 and P6 carrying the D835H mutation, primers P7 and P8 carrying the D835V mutation, and primers P9 and P10 carrying the D835F mutation were used to amplify the wild-type Tvec-FLT3-D835 plasmid. Then, the amplified fragments were fused and cloned into the pGem-T vector (Takara, China). In crRNA screening, a 351-bp DNA fragment harboring the D835region was amplified with primers P11 and P12 from the above recombinant plasmids, then purified and quantified as mentioned above. The plasmid templates of IDH2-R172K, EGFR-L858R. NRAS-G12D, and their WT forms were constructed in the same way. And the plasmid templates of EGFR-e19del (E746-A750 deletion) and its corresponding WT form were directly synthesized by GenScript (Nanjing, China). The nucleotide sequences of all primers are listed in Table 2.

TABLE 2
PCR and RPA primer sequences

| ID | Name | Sequence (SEQ ID NOS: 1-38) |
|---|---|---|
| P1 | FLT3-D835-675bp-For | GCCTCTCACTTTTGCTCGGA |
| P2 | FLT3-D835-675bp-Rev | AGGATTGCACTCAAAGGCCC |
| P3 | T-vec-D835Y-For | GGCTCGATATATCATGAGTGATTCC |
| P4 | T-vec-D835Y-Rev | CATGATATATCGAGCCAATCCAAAG |
| P5 | T-vec-D835H-For | GGCTCGACATATCATGAGTGATTCC |
| P6 | T-vec-D835H-Rev | CATGATATGTCGAGCCAATCCAAAG |
| P7 | T-vec-D835V-For | GGCTCGAGTTATCATGAGTGATTCC |
| P8 | T-vec-D835V-Rev | CATGATAACTCGAGCCAATCCAAAG |
| P9 | T-vec-D835F-For | GGCTCGATTTATCATGAGTGATTCC |
| P10 | T-vec-D835F-Rev | CATGATAAATCGAGCCAATCCAAAG |
| P11 | FLT3-D835-351bp-For | GGTACCTCCTACTGAAGTTG |
| P12 | FLT3-D835-351bp-Rev | GTAAGCAGACTGCTGTGAGG |
| P13 | FLT3-D835-RPA-F1 | AAAGTGGTGAAGATATGTGACTTTGGATTGGC |
| P14 | FLT3-D835-RPA-F2 | GGTGAAGATATGTGACTTTGGATTGGCTCG |
| P15 | FLT3-D835-RPA-F3 | CTCCAGGATAATACACATCACAGTAAATAACAC |
| P16 | FLT3-D835-RPA-R1 | CACAACACAAAATAGCCGTATAAAAATAAGTAGG |
| P17 | FLT3-D835-RPA-R2 | TTTACCATGATAACGACACAACACAAAATAGCC |
| P18 | FLT3-D835-RPA-R3 | CCTTTTAAGCATAAGTAAGCAGACTGCTGTGAGGG |
| P19 | IDH2-R172-506bp-For | CATGAAGAATTTTAGGACCC |
| P20 | IDH2-R172-506bp-Rev | CCAGCCTCACCTCGTCGGTG |
| P21 | T-vec-R172K-For | ATTGGCAAGCACGCCCATGG |
| P22 | T-vec-R172K-Rev | GGCGTGCTTGCCAATGGTGA |
| P23 | EGFR-L858-402bp-For | AGCCATAAGTCCTCGACGTG |
| P24 | EGFR-L858-402bp-Rev | CTGCGAGCTCACCCAGAATG |
| P25 | T-vec-L858R-For | TTTGGGCGGGCCAAACTGCTGG |
| P26 | T-vec-L858R-Rev | TTTGGCCCGCCCAAAATCTGTGATC |
| P27 | NRAS-G12-299bp-For | AATGGAAGGTCACACTAGGG |
| P28 | NRAS-G12-299bp-Rev | ACAGAATATGGGTAAAGATG |
| P29 | T-vec-G12D-For | GGAGCAGATGGTGTTGGGAA |
| P30 | T-vec-G12D-Rev | CAACACCATCTGCTCCAACC |
| P31 | IDH2-R172-RPA-F | TCCCTGGCTGGACCAAGCCCATCACCTTTGGC |
| P32 | IDH2-R172-RPA-R | TGCCCAGGTCAGTGGATCCCCTCTCCACCC |
| P33 | EGFR-e19-RPA-F | TCCCAGAAGGTGAGAAAGTTCAAATTCCCGTCGC |
| P34 | EGFR-e19-RPA-R | TTCAGAGCCATGGACCCCCACACAGCAAAGC |
| P35 | EGFR-L858-RPA-F | AACGTACTGGTGAAAACACCGCAGCATGTC |
| P36 | EGFR-L858-RPA-R | CACCTCCTTACTTTGCCTCCTTCTGCATGG |
| P37 | NRAS-G12-RPA-F | TTCTTGCTGGTGTGAAATGACTGAGTACAAACTG |
| P38 | NRAS-G12-RPA-R | TCTATGGTGGGATCATATTCATCTACAAAGTGG |

*Mutated bases are in bold. The underlined base T in primer P31 is an introduced mutation to form a TTTG PAM for the detection of IDH2-R172K. The underlined base C in primer P33 is an introduced mutation to destroy an unwanted SaqAI restriction site near the target EGFR-e19del site.

### CRISPR Reaction System

The Cas12a-based detection was performed according to a previous description with modifications (Wang X et al. Commun Biol 3, 62 (2020). [PubMed: 32047240]). Briefly, crRNAs were designed according to the target sequences and synthesized by GenScript (Nanjing, China). The nucleotide sequences of all crRNAs are listed in Table 3. Cas12a protein was expressed and purified as described previously (Creutzburg SCA et al. Nucleic Acids Res 48, 3228-3243 (2020). [PubMed: 31989168]). The 20 μl reaction system included Cas12a (200ng/μl, 1 μl), crRNA (100 nM, 1 μl), 10× NEBuffer 3.1 (2 μl, NEB, MA, USA), RNase inhibitor (1 μl, Novoprotein, China), ssDNA-FQ reporter (25 μM, 1 μl, Genewiz, NJ, USA), an appropriate amount of PCR product or 5 μl RPA product to test, and supplementary ddH₂O. After sufficient mixing on the vortex shaker, the mixture was incubated at 37° C. for 20 min, and then the green fluorescence signal was visualized under a 485-nm blue lamp (Sangon, Shanghai, China). Fluorescence kinetics were monitored using a monochromator with excitation at 485 nm and emission at 520 nm.

TABLE 3
crRNA sequences

| Name | Sequence (SEQ ID NOS: 39-60) |
|---|---|
| FLT3-D835Y-crRNA1 | UAAUUUCUACUAAGUGUAGAUGAUUGGCUCGAUAUAUCAUGAGU |
| FLT3-D835Y-crRNA2 | UAAUUUCUACUAAGUGUAGAUGAUUGGCUCGAUACAUCAUGAGU |
| FLT3-D835Y-crRNA3 | UAAUUUCUACUAAGUGUAGAUGAUUGGCUCUAUAUAUCAUGAGU |
| FLT3-D835Y-crRNA4 | UAAUUUCUACUAAGUGUAGAUGAUUGGCUCGAUAUAUAAUGAGU |
| FLT3-D835H-crRNA1 | UAAUUUCUACUAAGUGUAGAUGAUUGGCUCGACAUAUCAUGAGU |
| FLT3-D835H-crRNA2 | UAAUUUCUACUAAGUGUAGAUGAUUGGCUCGACACAUCAUGAGU |
| FLT3-D835V-crRNA1 | UAAUUUCUACUAAGUGUAGAUGAUUGGCUCGAGUUAUCAUGAGU |
| FLT3-D835V-crRNA2 | UAAUUUCUACUAAGUGUAGAUGAUUGGCUCGCGUUAUCAUGAGU |
| FLT3-D835V-crRNA3 | UAAUUUCUACUAAGUGUAGAUGAUUGGCUCGAGUUCUCAUGAGU |
| FLT3-D835F-crRNA | UAAUUUCUACUAAGUGUAGAUGAUUGGCUCGAUUCAUCAUGAGU |
| FLT3-D835WT-crRNA1 | UAAUUUCUACUAAGUGUAGAUGAUUGGCUCGAGAUAUCAUGAGU |
| FLT3-D835WT-crRNA2 | UAAUUUCUACUAAGUGUAGAUGAUUGGCUCGAGACAUCAUGAGU |
| FLT3-D835WT-crRNA3 | UAAUUUCUACUAAGUGUAGAUGAUUGGCUCUAGAUAUCAUGAGU |
| FLT3-D835WT-crRNA4 | UAAUUUCUACUAAGUGUAGAUGAUUGGCUCGAGAUAUAAUGAGU |
| IDH2-172WT-crRNA | UAAUUUCUACUAAGUGUAGAUGCAGGCGCGCCCAUGGCGACCAG |
| IDH2-R172K-CRRNA | UAAUUUCUACUAAGUGUAGAUGCAAGCGCGCCCAUGGCGACCAG |
| EGFR-e19WT-crRNA | UAAUUUCUACUAAGUGUAGAUGGAGAUGUUGCUUCUCUUAAUUC |
| EGFR-e19del-crRNA | UAAUUUCUACUAAGUGUAGAUGGAGAUGUUUUGAUAGCGACGGG |
| EGFR-858WT-crRNA | UAAUUUCUACUAAGUGUAGAUGGCUGGCUAAACUGCUGGGUGCG |
| EGFR-L858R-crRNA | UAAUUUCUACUAAGUGUAGAUGGCGGGCUAAACUGCUGGGUGCG |
| NRAS-12WT-crRNA | UAAUUUCUACUAAGUGUAGAUCCAACACCACCCGCUCCAACCAC |
| NRAS-G12D-crRNA | UAAUUUCUACUAAGUGUAGAUCCAACACCAUCCACUCCAACCAC |

*Target sequences are underlined, mutated bases are in bold, and introduced mismatches are in italic.

### Recombinase Polymerase Amplification

Isothermal amplification of plasmids or patient genomic DNA was conducted by the GenDx ERA Kit (Suzhou GenDx Biotech, China). For CRISPR detection, the 50 μl RPA reaction system included 20 μl reaction buffer, 11 μl ERA basic buffer, 2.5 μl forward primer (10 nM), 2.5 μl reverse primer (10 nM), 2 μl DNA template, 2 μl activator, and supplementary ddH₂O. For the Method of the disclosure, an additional 2 μl restriction enzyme was mixed into the above RPA system. Fastcut-EcoRV (Monad, Suzhou, China), FastDigest-BseLI, FastDigest-SaqAI, FastDigest-Mscl, and FastDigest-Bvel (Thermo Fisher Scientific, MA, USA) were used in Method detection of FLT3-D835Y, IDH2-R172K, EGFR-e19del, EGFR-L858R, and NRAS-G12D, respectively. Then, the mixture was incubated at 37° C. for 20 min. After RPA, 5 μl of the amplification product was transferred to the crRNA-guided Cas12a reaction. The primers for RPA are listed in Table 2.

### First-Generation Sequencing and Next-Generation Sequencing

For FGS, 25 μl PCR products or 25 μl RPA products were purified using an Axy Prep PCR Clean-up Kit (Axygen, CA, USA) and quantified by a Nanodrop2000 (Thermo Fisher Scientific, MA, USA). For each sample, approximately 300 ng amplified DNA fragments were sent to FGS by Tsingke (Beijing, China). For NGS, different barcoded primers were used to amplify the FLT3-D835 region of different samples. The PCR products were purified and mixed equally for NGS by the Illumina NextSeq 500 (2 x 150) platform at the CAS-MPG Partner Institute for Computational Biology Omics Core, Shanghai, China. The primers for NGS are listed in Table 4.

TABLE 4
Next-generation sequencing primer sequences

| Name | Sequence (SEQ ID NOS: 61-89) |
|---|---|
| D835-DSF1 | ATCACGTCACCGGTACCTCCTACTGA |
| D835-DSF2 | CGATGTTCACCGGTACCTCCTACTGA |
| D835-DSF3 | TTAGGCAGTCACCGGTACCTCCTACTGA |
| D835-DSF4 | TGACCAGTCACCGGTACCTCCTACTGA |
| D835-DSF5 | ACAGTGCTTCACCGGTACCTCCTACTGA |
| D835-DSF6 | GCCAATTCACCGGTACCTCCTACTGA |
| D835-DSF7 | CAGATCTTCACCGGTACCTCCTACTGA |
| D835-DSF8 | ACTTGAAATCACCGGTACCTCCTACTGA |
| D835-DSF9 | GATCAGGTCACCGGTACCTCCTACTGA |
| D835-DSF10 | TAGCTTCCTCACCGGTACCTCCTACTGA |
| D835-DSF11 | GGCTACTCACCGGTACCTCCTACTGA |
| D835-DSF12 | CTTGTATCACCGGTACCTCCTACTGA |
| D835-DSF13 | AGTCAATCACCGGTACCTCCTACTGA |
| D835-DSF14 | AGTTCCTTCACCGGTACCTCCTACTGA |
| D835-DSF15 | ATGTCAGCTCACCGGTACCTCCTACTGA |
| D835-DSF16 | CCGTCCATTCACCGGTACCTCCTACTGA |
| D835-DSF17 | GTAGAGCTCACCGGTACCTCCTACTGA |
| D835-DSF18 | GTCCGCATCACCGGTACCTCCTACTGA |
| D835-DSF19 | GTGAAATCTCACCGGTACCTCCTACTGA |
| D835-DSF20 | GTGGCCTCACCGGTACCTCCTACTGA |
| D835-DSF21 | GTTTCGTCACCGGTACCTCCTACTGA |
| D835-DSF22 | CGTACGGTCACCGGTACCTCCTACTGA |
| D835-DSF23 | GAGTGGAGTCACCGGTACCTCCTACTGA |
| D835-DSF24 | GGTAGCATCACCGGTACCTCCTACTGA |
| D835-DSF25 | ACTGATGTCACCGGTACCTCCTACTGA |
| D835-DSF26 | ATGAGCCATCACCGGTACCTCCTACTGA |
| D835-DSF27 | ATTCCTTCACCGGTACCTCCTACTGA |
| D835-DSF28 | CACCGGCATCACCGGTACCTCCTACTGA |
| D835-DFR | GAAATAGCAGCCTCACATTGCC |

### TaqMan qPCR

TaqMan qPCR probes and primers were designed and synthesized by Tsingke (Beijing, China). The probes were the complementary sequence to the FLT3-D835Y template with 5′ reporter dye FAM and 3′ MGB. The 20 μl qPCR system included 2× Taq Pro HS Universal Probe Master Mix (10 μl, Vazyme, Nanjing, China), qPCR-F (10 μM×0.4 μl), qPCR-R (10 μM×0.4 μl), TaqMan probe (10 μM×0.2 μl), Template DNA (1 μl), and ddH₂O (8 μl). PCR cycling conditions were 95° C. for 30 s and 45 cycles of 95° C. for 10 s and 60° C. for 30 s. The sequences of qPCR primers and probes are listed in Table S4. Commercial kits performed the qPCR detection of EGFR-e19del, EGFR-L858R and NRAS-G12D mutations.

### Statistical Analysis

All experiments were repeated three times. Statistical analyses were carried out with GraphPad Prism 8.0. Unpaired two-tailed Student's t-test was used for comparison between two groups. Quantitative data are expressed as mean value±standard error. * P<0.05, ** P<0.01, *** P<0.001, **** P<0.0001: ns., no significance.

### Example 1: Validation by Detecting Drug-Resistant FLT3-D835 Mutations

As proof-of-concept, we used Method herein to detect the drug-resistant FLT3-D835 mutations in acute myeloid leukemia (AML), which includes four major mutations according to the cBioportal database: D835Y (c.2503G>T) with a frequency of 45%, D835H (c.2503G>C) with 22%, D835V (c.2504A>T) with 14%, and D835F (c.2503GA>TT) with less than 1% (FIG. 1b). EcoRV restriction enzyme can recognize and digest WT D835 sequence (-GATATC-), while ignore D835 mutant sequences. We first tested the activity and specificity of EcoRV digestion in its special buffer using 100% WT and 100% D835Y/H/V/F PCR fragments. As expectation, WT D835 fragments were degraded completely, while D835Y/H/V/F fragments were resistant (FIG. 1c).

Next, to test the feasibility of EcoRV digestion in the RPA reaction, 5e10 copies of 100% WT and 100% D835Y/H/V/F PCR fragments were treated in the RPA mixture without primers, at 37° C. for 20 minutes, and then detected by Cas12a reaction. The fluorescence signal of WT D835 fragments was eliminated by EcoRV digestion (FIGS. 1d-e), while that of the D835Y/H/V/F fragments slightly enhanced for unknown reason (FIGS. 1d-e), indicating that EcoRV did not compromise RPA and suggesting that it can enhance the sensitivity of detecting rare D835Y/H/V/F mutations.

### Example 2: Enhancing Specificity with Optimized crRNAs

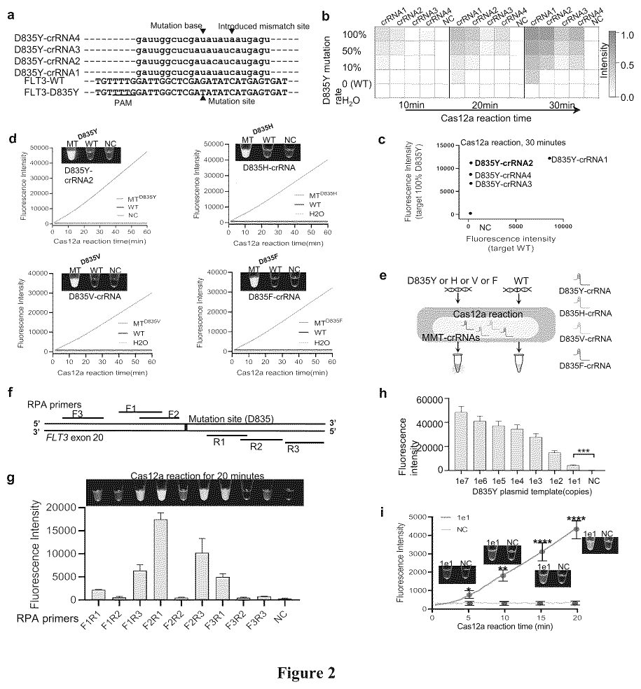

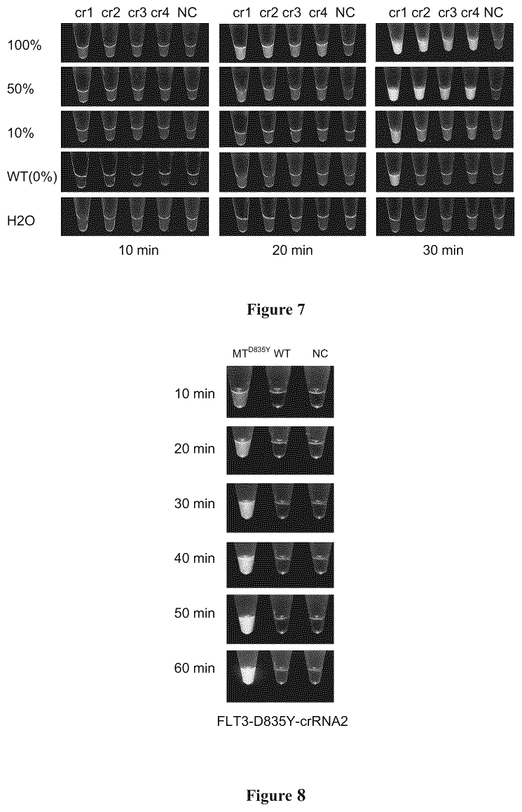

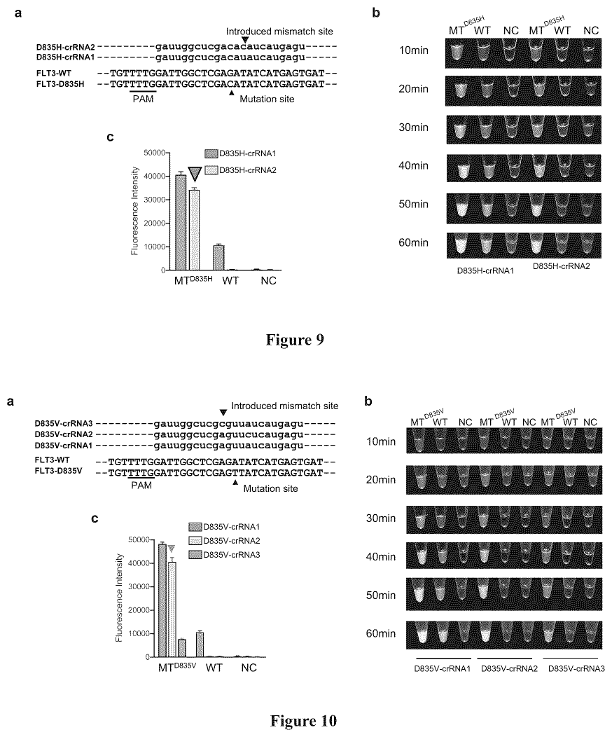

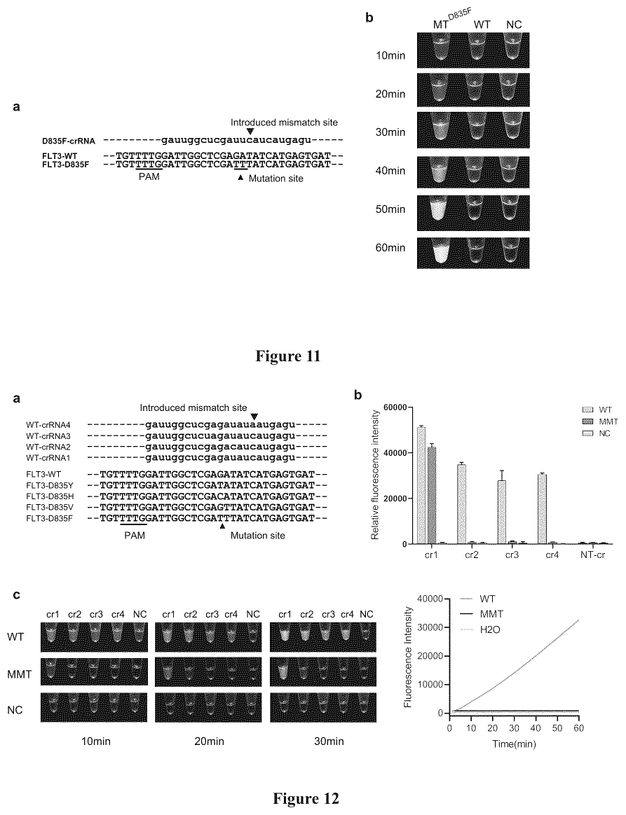

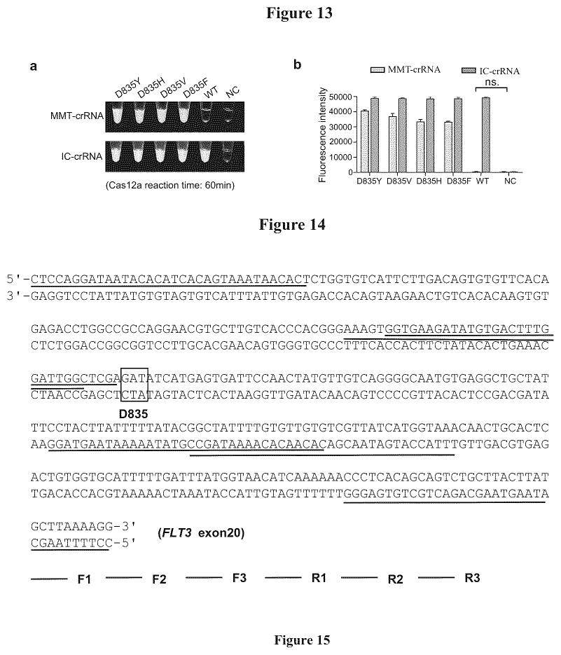

The specificity of crRNAs in the Cas 12a reaction determines the accuracy of CRISPR detection. To screen for optimal crRNA for detecting FLT3-D835Y, we designed four crRNAs (Table 3), with FLT3-D835Y-crRNA1 perfectly matching the mutant sequence and FLT3-D835Y-crRNA2-4 bearing various mismatches (FIG. 2a). We compared the sensitivity and specificity of these four crRNAs in detecting D835Y in PCR fragments (1e10 copies) including the D835Y and WT alleles, with the mutant constituting 100%. 50%. 10%, and 0% of the total DNA (FIG. 2b). After 30 minutes of Cas 12a reaction, all the 4 crRNAs, particularly crRNAl and crRNA2, could detect the sample with 100% D835Y, while crRNA1 but not crRNA2 produced strong signal even for WT sample (FIGS. 2b-c, FIG. 7). Furthermore, for crRNA2, lengthening the incubation (to 60 min) led to stronger fluorescence when detecting the 100% D835Y sample, but the WT allele (in the 100% WT sample) remained undetectable, crRNA2thus seemed to be the optimal crRNA for D835Y detection based on its excellent sensitivity and specificity (FIG. 2d, FIG. 8). Optimal crRNAs for D835H/V/F were similarly identified (FIG. 2d, FIGS. 9-11). We also identified crRNAs for WT and internal control (IC) cases (FIGS. 12-13).

To simplify the diagnosis of the four drug-resistant FLT3-D835 mutations, we pooled the D835Y/H/V/F crRNAs (MMT-crRNAs) and the 4 mutant templates into a single reaction, finding that it produced strong fluorescence signal (FIG. 2e), whereas the WT sample did not show any signal, as expected (FIG. 14).

### Example 3: Enhancing Sensitivity with Optimized RPA Primers

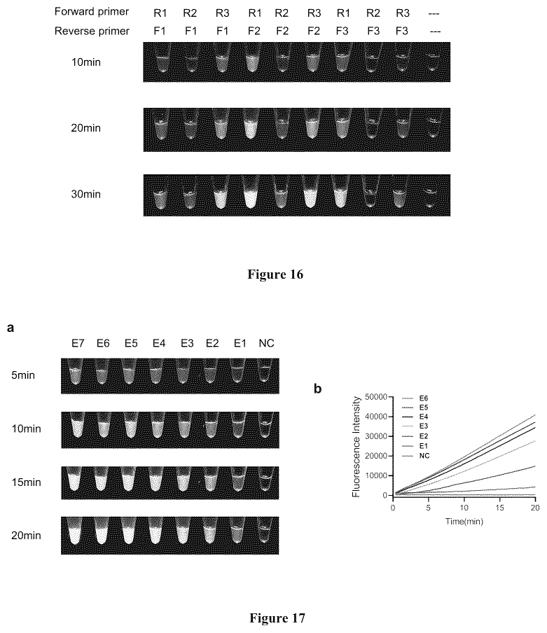

We next sought to improve method sensitivity by optimizing the RPA amplification efficiency. To this end, three forward (RPA-F1-3) and reverse (RPA-R1-3) primers (Table 2) were designed (FIG. 2f, FIG. 15) and tested on 1e2 copies of D835Y plasmid templates in standard RPA reaction (for 20 minutes at 37° C.). The amplified products were then detected by MMT-crRNAs-induced Cas12a reaction, which indicates that the F2R1 combination produced strongest signal (FIG. 2g, FIG. 16). Further analysis indicates that using F2R1, as low as 10 copies of plasmid templates could be detected (FIG. 2h, FIG. 17) only after 20 min of Cas12a reaction (FIG. 2i). Therefore, we chose F2 and R1 as the RPA primer pair and 20 min as the Cas12a reaction time in the subsequent experiments.

### Example 4: Method Achieves a 0.001% Sensitivity in FLT3-D835Y Detection

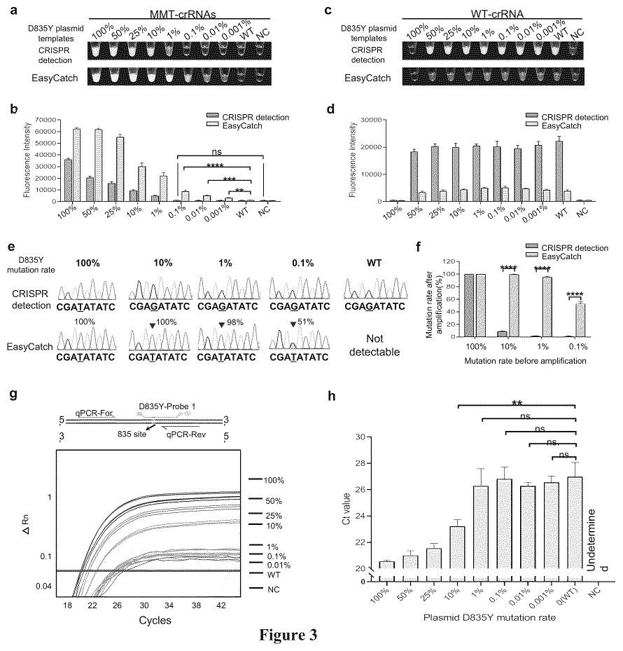

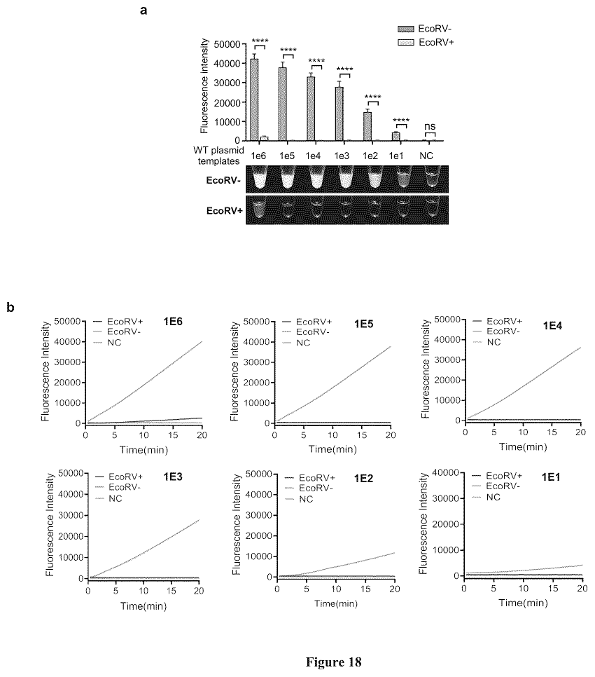

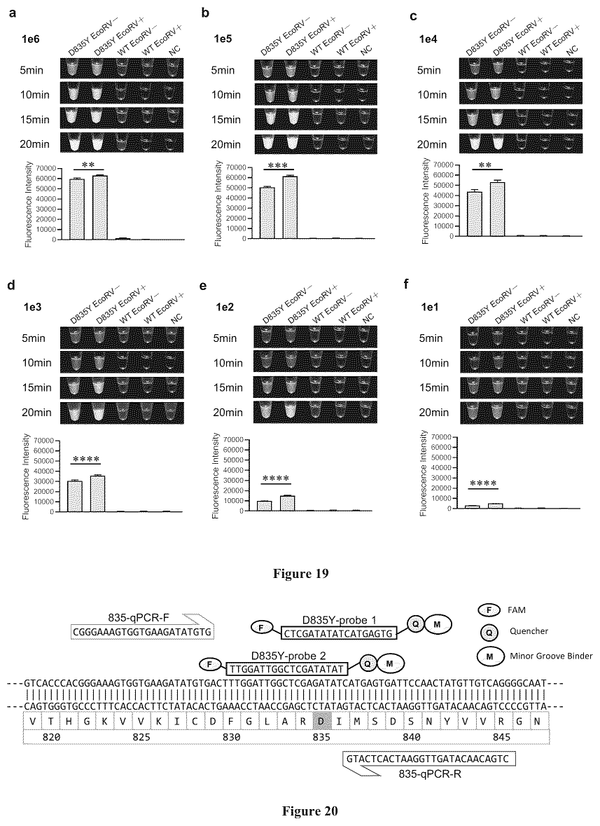

With the optimized gRNA and primers, we set out to determine the detection limit of Method for FLT3-D835Y. We first quantified the effect of EcoRV on WT sequence, finding that EcoRV could almost completely inhibit the amplification of up to 1e6 copies of templates (FIG. 18) but spared the mutant target as expected (FIG. 19). We then mixed the mutant and WT templates at various ratios, with FLT3-D835Y included 100%, 50%, 25%, 10%, 1%, 0.1%, 0.01%, and 0.001% of the total templates, and used 1e6 copies of the templates as the input for Method, finding the detection limit being 0.001% (i.e., 10 copies of D835Y template amid 999990 copies of WT template). In contrast, in the absence of EcoRV, the detection limit was 1000× lower (1%) (FIGS. 3a-b). WT inhibition effect was seen for WT-crRNA induced Cas12a reaction (FIG. 3c-d). Finally, FGS directly demonstrated that EcoRV markedly enriched the mutant allele in the RPA mixture from 10%, 1% and 0.1% to 100%, 98% and 51%, respectively (FIGS. 3e-f).

### Example 5: Method Outperformed qPCR-Based Assay for FLT3-D835Y Detection

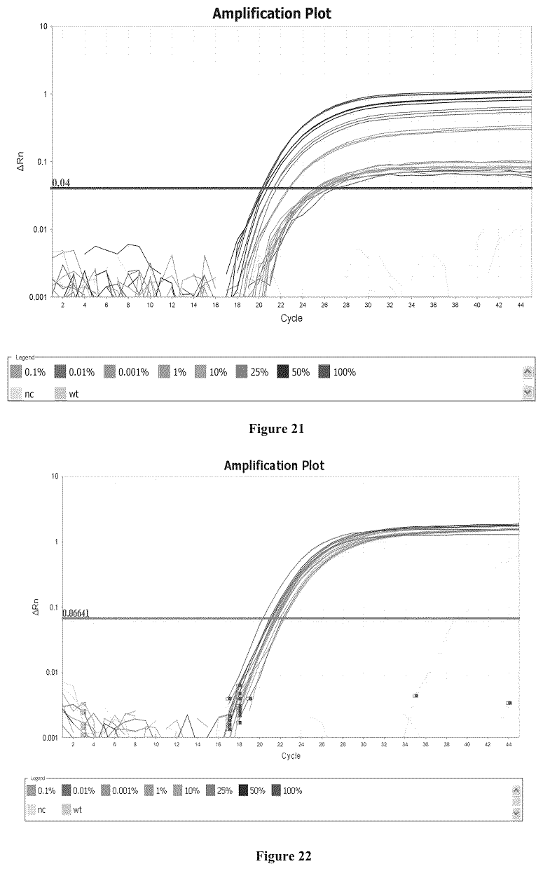

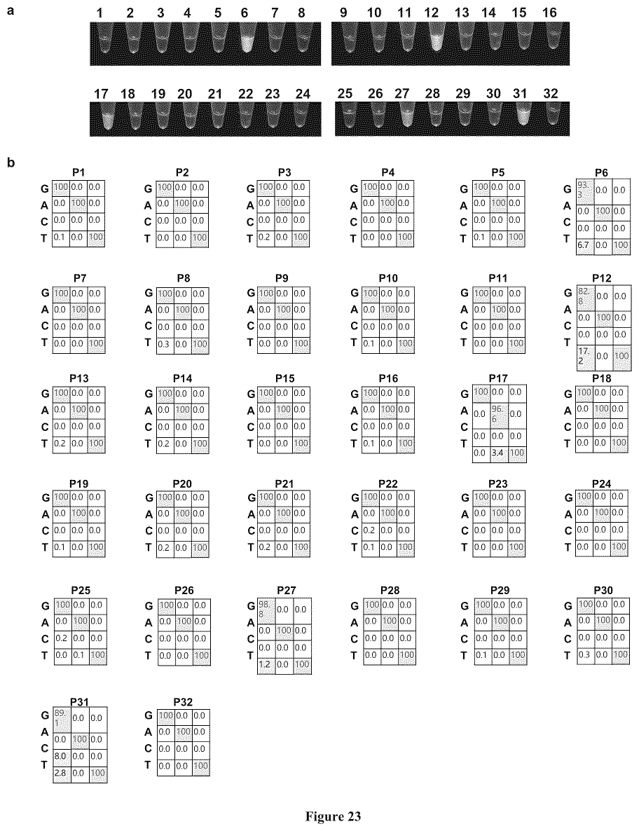

We next compared our method with the commonly used qPCR-based detection method. We designed two D835Y-specific TaqMan probes and a pair of qPCR primers (Table 5) for FLT3-D835Y detection (FIG. 20), then the two probes were tested using the samples above. The results showed that the amplification curves of different samples were mainly distinct in D835Y-probe 1, but not D835Y-probe 2-involved qPCR, indicating a higher specificity of probe 1. Thus we chose probe 1 for D835Y qPCR detection (FIGS. 21-22). The results of probe 1 showed that the amplification curves of 100%, 50%, 25%, and 10% mutant samples were gradually shifted to the right, consistent with the decrease in D835Y template amount. However, the amplification curves of 1%, 0.1%, 0.01% mutant samples cannot be distinguished from that of the WT sample (FIG. 3g). Comparing their Ct values also showed that D835Y-probe 1-involved qPCR could not distinguish between 1% mutant sample and WT sample, indicating its sensitivity is of only 1% ˜10% (FIG. 3h). Together, these results suggested that Method is much more sensitive than TaqMan qPCR in detecting FLT3-D835Y mutation.

TABLE 5
Primers and probes of Taqman qPCR

| Name | Sequence (SEQ ID NO: 90-93) |
|---|---|
| 835-qPCR-F | cgggaaagtggtgaagatatgtg |
| 835-qPCR-R | ctgacaacatagttggaatcactcatg |
| D835Y-probe 1 | FAM-ctcgaGatatcatgagtg-MGB |
| D835Y-probe 2 | FAM-ttggattggctcgaGatat-MGB |

*D835Y mutated bases were in bold.

### Example 6: Method Accurately Detected FLT3-D835Y/V/H/F Mutations in Clinical Samples

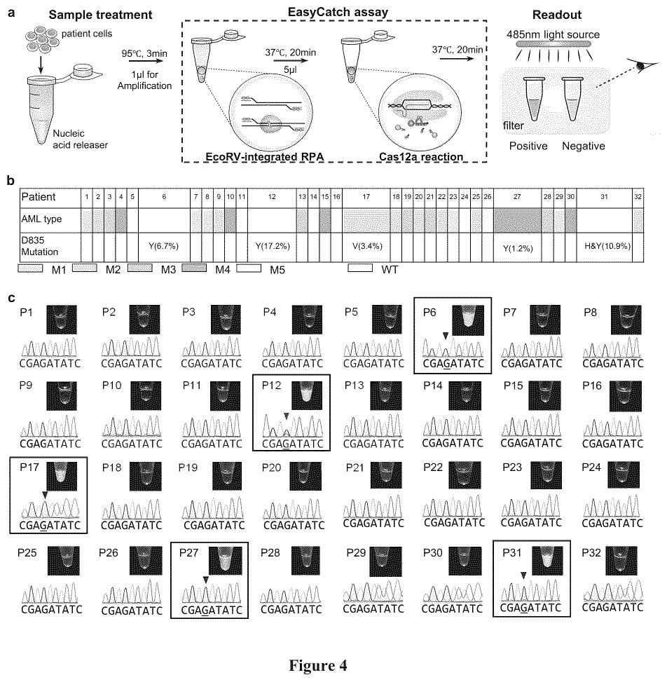

After the tests on plasmid templates, frozen cell samples of AML patients were used for D835Y/V/H/F mutation detection. Briefly, genomic DNA of patient cells was released by a nucleic acid releaser and then treated by the present method. Finally, the results were visualized with naked eyes under a blue lamp (FIG. 4a). The FLT3 gene mutation status of these samples had been analyzed by NGS before, and P6, P12, P17, P27, and P31 carried drug-resistant D835Y/V/H/F mutations with the mutation rates of 6.7% (Y), 17.2% (Y), 3.4% (V), 1.2% (Y), 10.9% (H and Y), respectively (FIG. 4b, FIG. 23). Then, we applied our method and FGS to detect the mutation of these 32 AML cell samples. The results showed that all five mutant samples were successfully identified by the present method, but only two samples with relatively high mutation rates, P12 of 17.2% and P31 of 10.9%, were identified by FGS (FIG. 4c, FIG. 23). Thus, our method is applicable to clinical samples.

### Example 7: Method Enables Clinical Diagnosis in an Hour

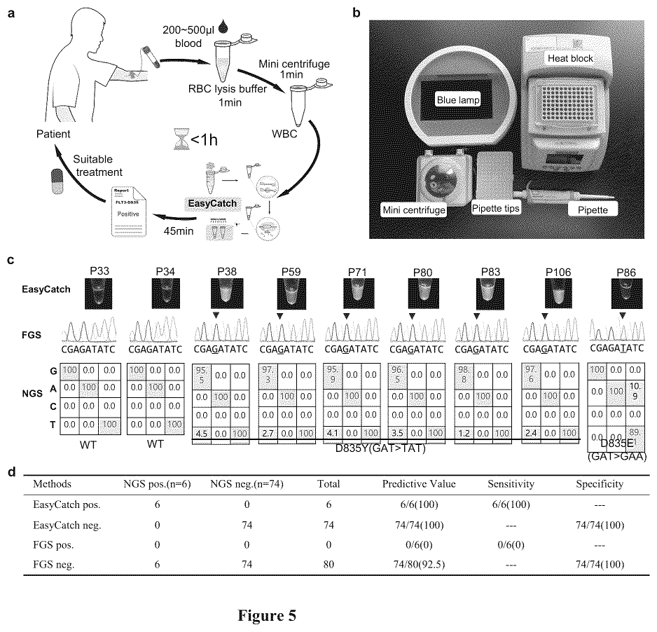

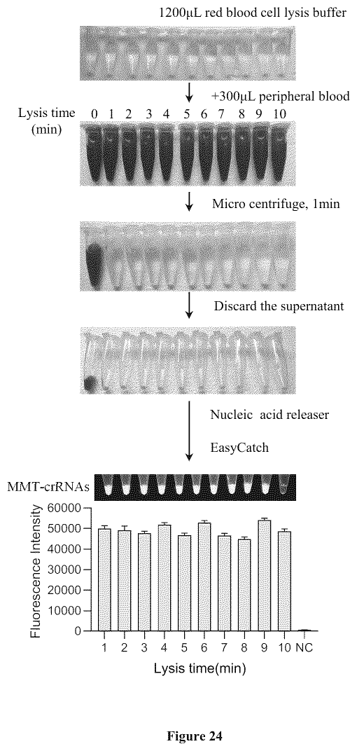

Considering clinical detection of drug-resistant mutation by NGS is time-consuming, we aim to further simplify the whole Method diagnosis process of drug-resistant FLT3-D835 mutations. To this end, we developed a white blood cell (WBC) enrichment method to treat fresh peripheral blood drawn from AML patients (FIG. 24). Briefly, 200500 ill of blood was incubated with four times the volume of red blood cell (RBC) lysis buffer for 1 minute to eliminate RBCs without nuclei, and the WBC precipitate was obtained by 1 min of centrifugation. Then, the genomic DNA of these WBCs was released by a simple nucleic acid releaser and detected by our method as mentioned above. The diagnostic result can be accomplished in 45 minutes from drawing blood, and then the drugs sensitive to the mutations will be delivered to relevant patients to avoid ineffective FLT3 inhibitor treatment. In this way, the whole process from drawing blood to making treatment decisions can be completed within 1 hour (FIG. 5a). In one embodiment, our method is simple and economical, and only needs a mini centrifuge, a 20 ill pipette and tips, a heat blocker, and a blue lamp with a 485-nm wavelength (FIG. 5b).

### Example 8: Method Outperforms FGS for Clinical Sample Analysis

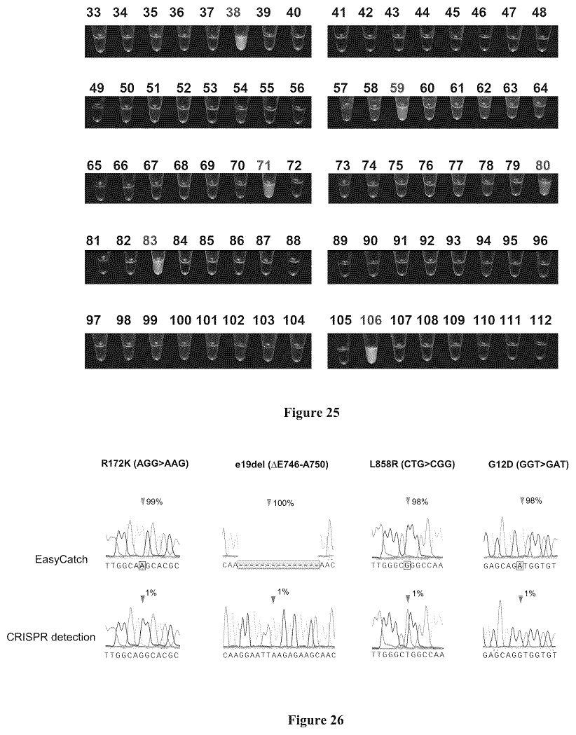

We next benchmarked our method against the commonly used FGS for detecting FLT3-D835 mutations in 80 AML patients (P33-P112) with unknown FLT3-D835 mutation status, with the samples also analyzed by NGS as the gold standard. Method, but not FGS, was able to detect D835Y in P38, P59, P71, P80, P83, and P106 (FIG. 5c, FIG. 25). NGS confirmed that six samples all harbored the D835Y mutation (at 4.5%, 2.7%, 4.1%, 3.5%, 1.2% and 2.4%, respectively). Notably, NGS showed that P86 carried 10.9% non-drug-resistant D835E (GAT>GAA) mutation. As this mutation did not produce a signal in our method, the data confirmed the high specificity of the method (FIG. 5c). The statistical analysis of the 80 cases showed that the present method is much more sensitive than FGS (100% vs. 0%) (FIG. 5d).

### Example 9: Method is Applicable to other Cancer Mutations

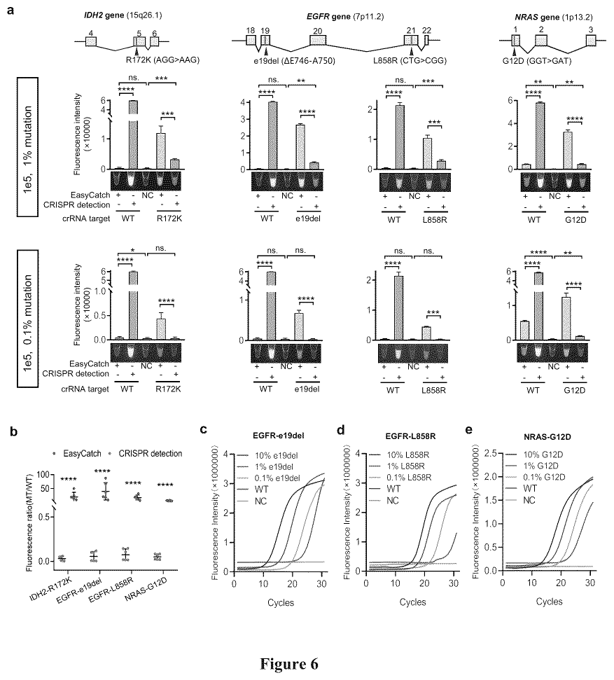

To verify the versatility of our method, we applied it to mutations at 4 other genes (IDH2, EGFR and NRAS). IDH2 R172K is a hotspot mutation in glioma and leukemia, and of prognostic and therapeutic value. EGFR e19del and L858R are the two main mutations sensitive to EGFR-TKIs, thus of great therapeutic value for patients with lung cancer. At the same time, the NRAS G12D is a driver mutation in leukemia and colorectal cancer. All four mutations are important testing items in the clinic. We first compared Method with the CRISPR detection method to detect 1e5 copies of plasmid templates of 1% or 0.1% mutation rate. The results showed that the WT fluorescence signals were strong while the signals of all the four mutations were weak in CRISPR detection. However, the WT signals were almost invisible while the mutation signals were significantly increased in the present method (FIG. 6a). The FGS results of the amplified products also confirmed the excellent mutation enrichment effect of Method. In one embodiment, after the restriction enzyme-integrated RPA, 1% mutation rate of IDH2-R172K. EGFR-e19del, EGFR-L858R, and NRAS-G12D increased to as high as 99%, 100%, 98%, and 98%, respectively (FIG. 26). Further analysis indicated that the MT/WT fluorescence ratios in the present method were hundreds of times higher than that of CRISPR detection (FIG. 6b).

### Example 10

We also detected EGFR-e19del, EGFR-L858R, and NRAS-G12D mutations using commercial kits based on fluorescence qPCR. The tested samples were 1e5 copies of plasmid templates with a mutation rate of 10%, 1%, 0.1%, and 0% (WT), respectively. The results of all three sites showed that, the amplification curves of different samples were gradually shifted to right, consistent with the decreased mutation rate. However, we noticed the strong fluorescence signals in WT samples (FIGS. 6c-e). In our detection, the WT signal was completely inhibited by restriction digestion and mutation-specific crRNA. Thus, the present method is a versatile and reliable method for detecting cancer gene mutations, especially of rare mutation rates.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The disclosure may be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes which come within the meaning and range of equivalency of the claims are therefore intended to be embraced therein.

1. A method for amplifying, enriching or detecting target nucleic acids with alternation(s) of interest at a specified site in a sample, comprising digesting or degrading non-target nucleic acids without the alternation(s) of interest at the specified site in the sample by exposing the non-target nucleic acids to one or more proteins having an activity of cleaving nucleic acid that recognizes the base(s) at the specified site before or during amplification; wherein the alternation in the target nucleic acid includes deletion, substitution and/or insertion of one or more base(s) at the specified site as compared to the sequence of the non-target nucleic acid; wherein the method for detecting further comprises a step of detecting the amplified target nucleic acid after digesting or degrading.

2. (canceled)

3. The method according to claim 1, wherein the protein having an activity of cleaving nucleic acid is selected from the group consisting of a restriction endonuclease, a Cas enzyme, an Ago enzyme, a ZFN enzyme, a TALEN enzyme and a functional complex thereof, such as Cas enzyme/sgRNA complex, Ago/gDNA complex, or Cas12a/crRNA.

4. The method according to claim 3, wherein:
the restriction endonuclease is selected from the group consisting of BclI, BsaBI, AclWI, Bst4CI, Xmil, BlsI, PspFI, CviKI-1, CviJI, MscI, EcoP15I, BspACI, BseLI, BstKTI, PspN4I, BspLI, NlaIV, Bmil, MaiI, RsnAI, BspEI, HpaII, MroI, Kpn2I, BcoDI, BstDEI, Bpu10I, BtsIMutI, BasBI, BtsCI, NspI, Fail, EcoRV and Btsal;
the Cas enzyme is selected from the group consisting of Cas 9, Cas 12, Cas 13 and Cas 14, especially SpCas9, SaCas9, HypaCas9, StlCas9, spCas9-NG, LbCas12a, spCas9-mut, and ScCas9;
the Ago enzyme is selected from a group consisting of pfAgo, cbAgo, LrAgo, pfAgo-mut, Apol, pfAgo, TtAgo and MjAgo.

5. The method according to claim 1, wherein exposing the non-target nucleic acids to the one or more proteins having an activity of cleaving nucleic acid is performed by adding the protein(s) in an amplification mixture for amplifying the target nucleic acids.

6. The method according to claim 1, wherein the amplification is selected from the group consisting of: helicase-dependent amplification (HAD), polymerase chain reaction (PCR), DNA ligase chain reaction (LCR), isothermal DNA amplification, QBeta RNA replicase, RNA transcription-based amplification reactions, loop-mediated isothermal amplification (LAMP), RT-LAMP, recombinase polymerase amplification (RPA), reverse transcription-recombinase polymerase amplification (RT-RPA), helicase-dependent amplification (HDA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), and isothermal multiple displacement amplification (IMDA).

7. The method according to claim 1, wherein the target nucleic acid is detected by DNA staining, nucleic acid amplification, spectrophotometry, sequencing, fluorescent probe hybridization, fluorescence resonance energy transfer, optical microscopy, electron microscopy, CRISPR-based detection, visual based detection, sensor-based detection, color detection, gold nanoparticle based detection, electrochemical detection, semiconductor-based sensing, or combinations thereof.

8. The method according to claim 1, wherein the amplified target nucleic acid is detected with one or more protein(s) capable of recognizing a specific nucleic acid sequence, or a functional complex thereof.

9. The method according to claim 1, wherein the mutation(s), proteins for cleavage, proteins for detection, and amplification method are those listed in each of the ID No. in the following Table:

| No. | mutation | Proteins for cleavage | Proteins for detection | amplification method |
|---|---|---|---|---|
| 1 | 1494C>T | spCas9 | LbCas12a | RT-RPA |
| 2 | 1555A>G | spCas9 | LbCas12a | RT-LAMP |
| 3 | c.3604G>A | spCas9 | FnCas12a | RPA |
| 4 | c.3512T>C | BelI | Lb5Cas12a | LAMP |
| 5 | c.3586C>A | pfAgo | HkCas12a | HAD |
| 6 | c.1397G>T | BsaBI | TsCas12a | SDA |
| 7 | c.1405G>A | AclWI | BbCas12a | NASBA |
| 8 | c.1406G>T | Cas | BoCas12a | TMA |
| 9 | c.1799_1800delinsAC | Bst4CI | Lb4Cas12a | NEAR |
| 10 | c.1799_1800delinsAT | cbAgo | LbuCas13a | RCA |
| 11 | c.1799T>A | LrAgo | LwaCas13a | MDA |
| 12 | c.1799_1800delinsAA | SaCas9 | LbaCas13a | RAM |
| 13 | c.1798_1799delinsAA | HypaCas9 | PprCas13a | cHDA |
| 14 | c.1798_1799delinsAG | St1Cas9 | HheCas13a | SMART |
| 15 | c.1415A>G | XmiI | EreCas13a | 3SR |
| 16 | c.G>A | BlsI | AsCas12a | NEAR |
| 17 | c.2156G>C | PspFI | LbCas12a | RPA |
| 18 | c.2155G>T | CviKI-1 | LbCas12a | LAMP |
| 19 | c.2155G>A | CviJI | AsCas12a | HAD |
| 20 | c.2573T>G | MscI | TsCas12a | SDA |
| 21 | c.2582T>A | spCas9 | BbCas12a | NASBA |
| 22 | c.2303G>T | EcoP15I | BoCas12a | TMA |
| 23 | c.2369C>T | spCas9-NG | Lb4Cas12a | LAMP |
| 24 | c.1978G>C | BspACI | LbuCas13a | GEAR |
| 25 | c.2264T>C | BseLI | LwaCas13a | IMDA |
| 26 | c.2033G>A | Lbcas12a | LbaCas13a | PCR |
| 27 | c.929C>T | BstKTI | spCas9 | 3SR |
| 28 | c.929C>A | PspN4I | pfAgo | NEAR |
| 29 | c.929_930delinsAT | BspLI | cbAgo | RPA |
| 30 | c.928T>G | NlaIV | LrAgo | LAMP |
| 31 | c.930C>A | BmiI | Cas12b | HAD |
| 32 | c.1976_1977delinsAG | MaiI | Cas12a-mut | SDA |
| 33 | c.1976T>A | spCas9-mut | AapCas12b | NASBA |
| 34 | c.1975G>C | pfAgo-mut | BrCas12b | TMA |
| 35 | c.2524G>A | RsnAI | AacCas12b | LAMP |
| 36 | c.3522C>A | Cas12 | FnCas12a | GEAR |
| 37 | c.3520T>C | ApoI | Lb5Cas12a | IMDA |
| 38 | c.3522C>G | Cas14 | HkCas12a | PCR |
| 39 | c.3586C>A | Cas9 | TsCas12a | RPA |
| 40 | 176delC | spCas9 | BbCas12a | LAMP |
| 41 | 35delG | pfAgo | BoCas12a | HAD |
| 42 | c.395G>A | TtAgo | Lb4Cas12a | SDA |
| 43 | c.419G>A | BspEI | LbuCas13a | NASBA |
| 44 | c.419G>T | HpaII | LwaCas13a | TMA |
| 45 | c.418C>T | MroI | LbaCas13a | LAMP |
| 46 | c.418C>G | Kpn2I | PprCas13a | GEAR |
| 47 | c.515G>A | MjAgo | HheCas13a | IMDA |
| 48 | c.1849G>T | spCas9 | EreCas13a | PCR |
| 49 | c.1848_1849delinsCT | ZFN | AsCas12a | 3SR |
| 50 | AGC>ACC | scCas9 | LbCas12a | NEAR |
| 51 | c.2447A>T | BcoDI | LbCas12a | RPA |
| 52 | c.35G>C | spCas9 | AsCas12a | LAMP |
| 53 | c.34G>T | spCas9-NG | TsCas12a | HAD |
| 54 | c.35G>A | spCas9-NG | BbCas12a | SDA |
| 55 | c.34G>C | spCas9-NG | BoCas12a | NASBA |
| 56 | c.34G>A | spCas9-NG | Lb4Cas12a | TMA |
| 57 | c.35G>T | spCas9-NG | LbuCas13a | LAMP |
| 58 | c.37G>T | spCas9-NG | LwaCas13a | PCR |
| 59 | c.38G>A | spCas9-NG | LbaCas13a | RPA |
| 60 | c.37G>A | spCas9-NG | spCas9 | LAMP |
| 61 | G>C | saCas9 | pfAgo | HAD |
| 62 | C>G | scCas9 | cbAgo | SDA |
| 63 | G>A | spCas9-NG | LrAgo | NASBA |
| 64 | G>C | TALEN | Cas12b | TMA |
| 65 | A>G | ZFN | Cas12a-mut | LAMP |
| 66 | T>C | Ago | AapCas12b | GEAR |
| 67 | c.1513A>T | BstDEI | BrCas12b | IMDA |
| 68 | c.1543T>A | Bpu10I | AacCas12b | PCR |
| 69 | c.183A>C | spCas9 | AacCas12b | RPA |
| 70 | c.181C>A | spCas9 | FnCas12a | LAMP |
| 71 | c.182A>T | spCas9 | Lb5Cas12a | RPA |
| 72 | c.182A>G | spCas9 | HkCas12a | LAMP |
| 73 | c.1633G>A | BtsIMutI | TsCas12a | PCR |
| 74 | c.3140A>G | BasBI | BbCas12a | RPA |
| 75 | 516 A>T | spCas9 | BoCas12a | LAMP |
| 76 | 315 AGC → ACA | SaCas9 | LbCas12a | RPA |
| 77 | T>G | spCas9 | LwaCas13a | LAMP |
| 78 | T>C | scCas9 | LbaCas13a | |
| 79 | 2168A>G | spCas9 | HheCas13a | TMA |
| 80 | c.1982A>T | BtsCI | EreCas13a | LAMP |
| 81 | c.1940A>T | NspI | AsCas12a | GEAR |
| 82 | c.1919A>T | FaiI | LbCas12a | IMDA |
| 83 | c.865A>T | BtsaI | LbCas12a | RPA |
| 84 | GAA>CAA | pfAgo | AsCas12a | RT-LAMP |
| 85 | GAT>TAT | EcoRV | LbCas12a | RPA. |

10. A kit for amplifying or enriching or detecting target nucleic acids with alternation(s) of interest at a specified site in a sample, comprising reagents for amplification of target nucleic acids with alternation(s) of interest at the specified site in the sample and reagents for digesting non-target nucleic acids without the alternation(s) of interest at the specified site in the sample; wherein the alternation in the target nucleic acid includes deletion, substitution and/or insertion of one or more base(s) at the specified site as compared to the sequence of the non-target nucleic acid.

11. The kit according to claim 10, wherein the reagents for digesting the nucleic acid includes a protein having an activity of cleaving nucleic acid is selected from the group consisting of a restriction endonuclease, a Cas enzyme, an Ago enzyme, a ZFN enzyme, a TALEN enzyme and a functional complex thereof, such as Cas enzyme/sgRNA complex, Ago/gDNA complex, or Cas 12a/crRNA.

12. The kit according to claim 10, wherein the reagents for amplification include reagents for performing any of helicase-dependent amplification (HAD), polymerase chain reaction (PCR), DNA ligase chain reaction (LCR), isothermal DNA amplification, QBeta RNA replicase, RNA transcription-based amplification reactions, loop-mediated isothermal amplification (LAMP), RT-LAMP, recombinase polymerase amplification (RPA), reverse transcription-recombinase polymerase amplification (RT-RPA), helicase-dependent amplification (HDA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), and isothermal multiple displacement amplification (IMDA).

13. The kit according to claim 10, wherein the kit further comprises reagent(s) used for detecting the target nucleic acids; preferably, the reagent(s) used for detecting the target nucleic acids include reagent(s) used in any of DNA staining, nucleic acid amplification, spectrophotometry, sequencing, fluorescent probe hybridization, fluorescence resonance energy transfer, optical microscopy, electron microscopy, CRISPR-based detection, visual based detection, sensor-based detection, color detection, gold nanoparticle based detection, electrochemical detection, semiconductor-based sensing.

14. The kit according to claim 12, wherein the reagent(s) used for detecting the target nucleic acids is one or more protein(s) capable of recognizing a specific nucleic acid sequence, or a functional complex thereof, including Cas enzyme, Ago enzyme, ZFN enzyme, TALEN enzyme, and functional complexes thereof.

15. The kit according to claim 10, wherein the kit comprises:
the protein for cleavage which is selected from a group consisting of spCas9, BclI, pfAgo, BsaBI, AclWI, Cas, Bst4CI, cbAgo, LrAgo, SaCas9, HypaCas9, StlCas9, Xmil, BlsI, PspFI, CviKI-1, CviJI, MscI, EcoP 15I, spCas9-NG, BspACI, BseLI, Lbcas 12a, BstKTI, PspN4I, BspLI, NlaIV, Bmil, MaiI, spCas9-mut, pfAgo-mut, RsnAI, Cas12, Apol, Cas 14, Cas9, TtAgo, BspEI, HpaII, Mrol, Kpn21, MjAgo, ZFN, scCas9, BcoDI, TALEN, Ago, BstDEI, Bpu10I, BtsIMutI, BasBI, BtsCI, NspI, Fail, Btsal and EcoRV; listed in any one of the ID No. in Table A and reagent(s) for the amplification method selected from a group consisting of RT-RPA, RT-LAMP, RPA, LAMP, HAD, SDA, NASBA, TMA, NEAR, RCA, MDA, RAM, CHDA, SMART, 3SR, GEAR, IMDA and PCR, and
the protein for detection selected from a group consisting of LbCas12a, FnCas12a, Lb5Cas12a, HkCas12a, TsCas12a, BbCas12a, BoCas12a, Lb4Cas12a, LbuCas13a, LwaCas13a, LbaCas13a, PprCas13a, HheCas13a, EreCas13a, AsCas12a, TsCas12a, BbCas12a, BoCas 12a, Lb4Cas12a, spCas9, pfAgo, cbAgo, LrAgo, Cas12b, Cas12a-mut, Cas12b-mut, AapCas12b, BrCas12b, CcaCas13b, PsmCas 13b and AacCas 12b.

16. The method according to claim 8, wherein the protein(s) capable of recognizing a specific nucleic acid sequence include Cas enzyme, Ago enzyme, ZFN enzyme, TALEN enzyme, and functional complexes thereof.

17. The method according to claim 16, wherein:
the Cas enzyme is selected from the group consisting of Cas 9, Cas 12, Cas 13 and Cas 14, including SpCas9, SaCas9, HypaCas9, StlCas9, spCas9-NG, LbCas12a, spCas9-mut, and ScCas9;
the Ago enzyme is selected from the group consisting of pfAgo, cbAgo, LrAgo, pfAgo-mut, Apol, pfAgo, TtAgo and MjAgo;
the functional complex is selected from the group consisting of Cas enzyme/sgRNA complex, Ago/gDNA complex and Cas12a/crRNA complex, including a Cas9/sgRNA complex, a spCas9/sgRNA complex, a pfAgo/gDNA complex, and a LbCas12a/crRNA complex; or
the protein capable of recognizing a specific nucleic acid sequence is selected from the group consisting of LbCas12a, FnCas12a, Lb5Cas12a, HkCas12a, TsCas12a, BbCas12a, BoCas12a, Lb4Cas12a, LbuCas13a, LwaCas13a, LbaCas13a, PprCas13a, HheCas13a, EreCas13a, AsCas12a, TsCas12a, BbCas12a, BoCas12a, Lb4Cas12a, spCas9, pfAgo, cbAgo, LrAgo, Cas12b, Cas12a-mut, Cas12b-mut, AapCas12b, BrCas12b, CcaCas13b, PsmCas13b and AacCas12b, or functional complexes thereof.

18. The kit according to claim 11, wherein:
the restriction endonuclease is selected from the group consisting of BclI, BsaBI, AclWI, Bst4CI, Xmil, BlsI, PspFI, CviKI-1, CviJI, MscI, EcoP15I, BspACI, BseLI, BstKTI, PspN4I, BspLI, NlaIV, Bmil, MaiI, RsnAI, BspEI, HpaII, Mrol, Kpn2I, BcoDI, BstDEI, Bpu10I, BtsIMutI, BasBI, BtsCI, NspI, Fail, EcoRV and Btsal;
the Cas enzyme is selected from the group consisting of Cas 9, Cas 12, Cas 13 and Cas 14, especially SpCas9, SaCas9, HypaCas9, StlCas9, spCas9-NG, LbCas12a, spCas9-mut, and ScCas9;
the Ago enzyme is selected from a group consisting of pfAgo, cbAgo, LrAgo, pfAgo-mut, Apol, pfAgo, TtAgo and MjAgo.

19. The kit according to claim 14, wherein:
the Cas enzyme is selected from the group consisting of Cas 9, Cas 12, Cas 13 and Cas 14, including SpCas9, SaCas9, HypaCas9, St1Cas9, spCas9-NG, LbCas12a, spCas9-mut, and ScCas9;
the Ago enzyme is selected from the group consisting of pfAgo, cbAgo, LrAgo, pfAgo-mut, ApoI, pfAgo, TtAgo and MjAgo;
the functional complex is selected from the group consisting of Cas enzyme/sgRNA complex, Ago/gDNA complex and Cas12a/crRNA complex, including a Cas9/sgRNA complex, a spCas9/sgRNA complex, a pfAgo/gDNA complex, and a LbCas12a/crRNA complex; or the protein capable of recognizing a specific nucleic acid sequence is selected from the group consisting of LbCas12a, FnCas12a, Lb5Cas12a, HkCas12a, TsCas12a, BbCas12a, BoCas12a, Lb4Cas12a, LbuCas13a, LwaCas13a, LbaCas13a, PprCas13a, HheCas13a, EreCas13a, AsCas12a, TsCas12a, BbCas12a, BoCas12a, Lb4Cas12a, spCas9, pfAgo, cbAgo, LrAgo, Cas12b, Cas12a-mut, Cas12b-mut, AapCas12b, BrCas12b, CcaCas13b, PsmCas13b and AacCas12b, or functional complexes thereof.

20. The method according to claim 1, wherein the target nucleic acid with alternation(s) of interest is selected from a group consisting of FLT3 D835 mutations, IDH2 R172K, EGFR L858R, EGFR e19del, and NRAS G12D.

21. The kit according to claim 10, wherein the target nucleic acid with alternation(s) of interest is selected from a group consisting of FLT3 D835 mutations, IDH2 R172K, EGFR L858R, EGFR e19del, and NRAS G12D.
